(19) United States
(12) Patent Application Publication
(10) Pub. No.: US 2026/0308287 A1
(43) Pub. Date: Oct. 8, 2026

(54) PROCESS FOR THE PREPARATION OF POLYISOBUTENE FROM OLEFINS FROM ISOBUTANOL OBTAINED FROM MIXED GUERBET REACTION OF ETHANOL AND METHANOL

(71) Applicant: BASF SE, Ludwigshafen am Rhein (DE)

(72) Inventors: Svenja Wesp, Ludwigshafen am Rhein (DE); Markus Brym, Ludwigshafen am Rhein (DE); Thomas Wettling, Ludwigshafen am Rhein (DE); Oliver Feldmann, Ludwigshafen am Rhein (DE); Christian Prokop, Ludwigshafen am Rhein (DE); Marcel Bauer, Ludwigshafen am Rhein (DE); Alois Kindler, Ludwigshafen am Rhein (DE)

(73) Assignee: BASF SE, Ludwigshafen am Rhein (DE)

(21) Appl. No.: 19/482,025

(22) PCT Filed: May 8, 2024
(86) PCT No.: PCT/EP2024/062744

§ 371 Date: Nov. 6, 2025

(30) Foreign Application Priority Data
May 12, 2023 (EP) 23173203.3

(51) Int. Cl. C08F 10/10; B01J 6/00; B01J 23/89; B01J 37/04; C07C 1/24; C07C 7/04; C07C 11/09; C07C 29/34; C07C 29/80; C07C 31/12

(52) U.S. Cl. CPC C08F 10/10; B01J 6/001; B01J 23/8946; B01J 37/04; C07C 1/24; C07C 7/04; C07C 11/09; C07C 29/34; C07C 29/80; C07C 31/12

(57) ABSTRACT

A process manufactures polyisobutene by condensation of methanol and a second alcohol in the gas phase yielding an isobutanol-containing reaction mixture, dehydrating isobutanol to isobutene, followed by polymerization of the thus obtained isobutene to polyisobutene.

The present invention relates the mixed Guerbet reaction of ethanol and methanol to obtain isobutanol, subsequent dehydration so as to obtain isobutylene, and polymerization of the thus obtained isobutylene to polyisobutene.

In a preferred embodiment the present invention relates to the above-mentioned reaction wherein the catalyst for condensing alcohol Is comprises support material in contact with copper as promoter, wherein
a) the support material comprises hydrotalcite-like compounds,
b) the proportion of copper promoter is in the range of from 0.05 to 5.0% by weight,
c) the support material has an Mg/Al ratio in the range of from 90/10 to 70/30, where the ratio is based on the weight of the respective oxides, wherein the catalyst may optionally comprise at least one further promoter element from the group consisting of Pt, Rh, Ru, Pd, Co, Ni, Pd, Cu, Ag and Au and the content of further promoter is in the range 0.01 to 2% by weight, preferably 0.01 to 1% by weight, with the proviso that the amount of copper promoter is greater than the amount of further promoter; and a process for condensing alcohols by bringing an alcohol-comprising feed gas stream, comprising an alcoholic component selected from the group consisting of C₁-C₅alcohols, especially at least two different alcoholic components from the group consisting of C₁-C₅alcohols, into contact with the catalyst. The high performance of the process of the invention in respect of the high conversion, the good yields and selectivities to the target compound, preferably iso-butanol, contribute, in particular, to an economical process having a high efficiency.

### PRIOR ART

Low carbon alcohols such as C₃-C₄ alcohols are important chemical intermediates used for the production of various solvents, plasticizers, polymers, lubricants, surfactants, and personal care products (J. Mück, K. Kocik, M. Hajek, Z. Tisler, K. Frohlich, A. Kasparek; Transition metals promoting Mg—Al mixed oxides for conversion of ethanol to butanol and other valuable products: Reaction pathways; Applied Catalysis A: General, pp 118380, 2021). Currently, synthesis of lower alcohols depends on the OXO synthesis process which involves the hydroformylation of olefins followed by hydrogenation (J. Sun, Y. Wang; Recent Advances in Catalytic Conversion of Ethanol to Chemicals, ACS Catalysis, pp 1078-1090, 2014). However, this process has many disadvantages including the difficulty in the separation of catalysts and products, the use of noble metals, and the harsh reaction condition of high pressure. Additionally, it is desirable to shift from the use of fossil-based raw materials toward biobased raw materials e.g. ethanol due to their reduced carbon footprint.

One of the promising pathways for the production of “green” isobutanol involves the Guerbet reaction. Guerbet reaction has been investigated for the last 100 years for the production of higher alcohol where primary or secondary alcohol possessing a methylene group at the α-position is condensed with itself or another alcohol (N. Egan, M. Kumbhalkar, J. Buchnan, J. Dumesci, G. Huber; Chemistries and processes for the conversion of ethanol into middle-distillate fuels, Nature Reviews, pp 223-249 (3), 2019).

The Guerbet condensation of ethanol and methanol involves the following four main steps
1. Dehydrogenation of ethanol and methanol to acetaldehyde and formaldehyde
2. Aldol condensation of acetaldehyde and formaldehyde to produce propionaldehyde
3. Further aldol condensation of propionaldehyde with formaldehyde to isobutyraldehyde
4. Hydrogenation of isobutyraldehyde to isobutanol

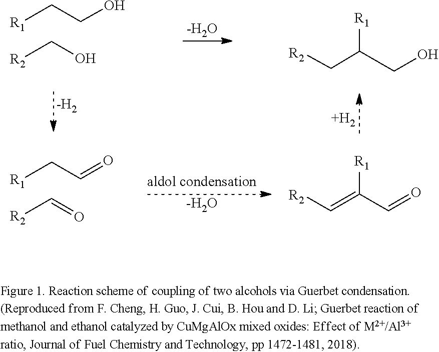

Various studies show the use of heterogeneous catalysts for the gas phase condensation of ethanol and methanol to either propanol or isobutanol. C. Carlini, M. Di Girolamo, A. Macinai, M. Marchionna, M. Noviello, A. Galletti, G. Sbrana; Selective synthesis of isobutanol by means of the Guerbet reaction: Part 2. Reaction of methanol/ethanol and methanol/ethanol/n-propanol mixtures over copper based/MeONa catalytic systems; Journal of Molecular Catalysis A: Chemical, pp 137-146, 2003 investigated the Guerbet condensation of ethanol/methanol and ethanol/methanol/propanol mixtures using sodium methoxide (MeONa) as a soluble basic component and copper-based heterogeneous catalysts.

J. Suarez, B. Subrmanim, R. Chaudhari; Vapor-phase methanol and ethanol coupling reactions on CuMgAl mixed metal oxides, Applied Catalysis A: General, pp 234-246, 2013 studied the effect of the catalyst composition of CuMgAlOx with varying Cu content on methanol and ethanol coupling reactions. Their observations indicated that MgAlOx without Cu deactivated rapidly while producing mainly C3+ alcohols at low alcohol conversions. Whereas in CuMgAlOx, C—C coupling (C3+ aldehydes, alcohols, and esters), non-C—C coupling (e.g., acetaldehyde, methyl formate, methyl acetate, ethyl acetate), and decomposition products (i.e., COx) were observed at significantly larger methanol and ethanol conversions and with minimal catalyst deactivation. Furthermore, D. Stošić, F. Hosoglu, S. Bennici, A. Travert, M. Capron, F. Dumeignil, J.-L.Couturier, J.-L. Duboise, A. Aurou; Methanol and ethanol reactivity in the presence of hydrotalcites with Mg/Al ratios varying from 2 to 7, Catalysis Communications, pp 14-18, 2017 confirmed the correlation between acidic and basic properties of hydrotalcite materials (Mg: Al ratio) to the reactivity of Guerbet condensation of ethanol and methanol. The highest alcohol conversions were reported for materials with the highest number of acidic and basic sites yielding high propanol and isobutanol selectivity. Cheng et. al., Journal of Fuel Chemistry and Technology, pp 1472-1481, 2018 showed the role of Cu species and alkalinity of CuMgAlOx mixed oxide catalysts towards Guerbet condensation of ethanol and methanol. Their results showed that the conversions of methanol and ethanol are strongly related to the surface areas of exposed Cu species and altered basicity.

U.S. Pat. No. 5,095,156 disclosed the conversion of methanol and ethanol over MgO or MgO admixed with charcoal. The reaction was carried out between sub-atmospheric pressure to up to 10 bar yielding the majority C3 and C4 alcohols in addition with COx and paraffin.

U.S. Pat. No. 5,559,275 relates to a process for the production of branched C4+ oxygenates from lower alcohols such as methanol, ethanol, propanol and mixtures thereof. The process comprises contacting the lower alcohols with a solid catalyst comprising a mixed metal oxide support having components selected from the group consisting of oxides of zinc, magnesium, zirconia, titanium, manganese, chromium, and lanthanides, and an activation metal selected from the group consisting of Group VIII metal, Group IB metals, and mixtures thereof.

U.S. Pat. No. 5,770,541 discloses a catalyst for the synthesis of isobutanol. In the catalyst, a noble metal is present on a support and the support has a first phase composed of crystallites of a mixed oxide comprising zirconium, manganese, zinc and a second phase having zirconium-doped hetaerolite particles comprising manganese and zinc. In addition, the support can also comprise a third phase composed of manganese, zinc and zirconium. A characteristic of the catalyst is that the crystallites of the first phase have a size in the range from 40 Å to 100 Å, the crystallites of the second phase have a size in the range from 200 Å to greater than 2000 Å and the crystallites of the third phase have a size in the range from 1000 Å to greater than 4000 Å.

U.S. Pat. No. 7,705,192 disclosed a process for making an isobutanol-containing product by contacting a reactant comprising ethanol and methanol over a catalyst wherein said reaction temperature is from about 200° C. to about 500° C. and said pressure is from about 0.1 MPa to about 20.7 MPa. The catalyst was derived from hydrotalcite of formula (M²⁺1-xM³⁺x(OH)₂)(An−x/n). yH₂O wherein M²⁺ is divalent Mg, or a combination of divalent Mg and at least one divalent member selected from the group consisting of Zn, Ni, Pd, Pt, Co, Fe, and Cu; M³⁺ is trivalent Al, or a combination of trivalent Al and at least one trivalent member selected from the group consisting of Fe and Cr.

In WO2009/097310A1 discloses a process for the catalytic reaction of ethanol in the presence of hydrogen. The process is based on catalysts which comprise thermally decomposed hydrotalcite, and the catalyst synthesis is carried out in the presence of EDTA (ethylenediaminetetraacetic acid) as complexing agent. The examples disclose the production of catalysts having cobalt as active metal.

According to WO2009/026506A1 hydrotalcites containing the anion of ethylenediaminetetraacetic acid are partially or fully thermally decomposed to provide catalysts useful for the conversion of ethanol and methanol to a reaction product comprising isobutanol.

CN105562046B discloses flower-shaped mesoporous hydroxyl Apatite catalysts with Ca/P/Sr/Cu active metal oxides to produce propyl alcohol through Guerbet condensation of ethanol and methanol.

WO2012035772A1 (EP2616418A1) relates to a method for producing an alcohol by a Guerbet reaction, wherein the reaction is performed in a gas phase and at a total pressure of less than 1 atm, using one or more raw material alcohols. The method involves the use of a basic catalyst which preferably comprises an apatite structure compound, such as, for example, calcium hydroxyapatite, strontium hydroxyapatite, hydrotalcite, MgO, Mg (OH) 2, and alkali metal supported-zeolite.

CN105562046B relates to a catalyst for condensing methanol and ethanol to prepare propyl alcohol and butanol. The catalyst uses flower-shaped mesoporous hydroxyapatite as the carrier, and oxide is loaded on the surface. The oxides are copper oxide and strontium oxide. The mole ratio of Ca to P to Sr to Cu is 1.65-1.9:1:0.019-0.089:0.018-0.067.

C. Carlini et al. report carrying out the Guerbet condensation using bifunctional catalysts based on magnesium- and aluminum-comprising mixed oxides (see C. Carlini et al., Journal of Molecular Catalysis A: Chemical 232 (2005) 13-20). The catalysts can comprise the elements Pd, Rh, Ni and Cu as active metals. Furthermore, it is stated that the magnesium- and aluminum-comprising mixed oxides used for producing the catalysts have a hydrotalcite structure.

Marcu et al. describe the conversion of ethanol into butanol by means of mixed oxide catalysts which are produced from magnesium- and aluminum-comprising hydroxides having a double layer structure (see loan-Cezar Marcu, Nathalie Tanchoux, Francois Fajula, Didier Tichit, Catal. Lett (2013) 143, p. 23-30). The catalysts can comprise the metals of the group consisting of Pd, Ag, Mn, Fe, Cu, Sm, Yb as active metals.

A publication by Di Cosimo et al. describes the use of catalysts based on magnesium- and aluminum-comprising mixed oxides for the condensation of alcohols (see J. I. Di Cosimo, Journal of Catalysis (2000) 190, p. 261-275). The publication presents a fundamental study on the influence of the support oxides on the catalytic reaction processes in the dimerization reaction.

Carlini et al., Journal of Molecular Catalysis A: Chemical 232 (2005) pages 13-20 report the synthesis of isobutyl alcohol from methanol and n-propanol through the guerbet condensation using bifunctional heterogeneous systems based on a dehydrogenating/hydrogenating metal (Pd, Rh, Ni or Cu) and a basic Mg—Al mixed oxide derived from hydrotalcite-type (HT) precursors.

F. Storgards et al., Topics in Catalysis, 61 (2018), pages 1888-1900 disclose (Table 1) a catalyst comprising 5 wt % Cu supported on hydrotalcite (Mg/Al ratio 2.25). The catalyst was prepared by coprecipitation of the corresponding metal nitrates. The amounts of Mg and Al were selected to give the same Mg/Al ratio as in the commercial HT, ca. 2.25. It was the aim to enhance the one-pot production of Guerbet alcohol (2BO (=2-butyl-2-octenal) from hexanol with the aid of heterogeneous catalysis.

K. A. Goulas et al., Journal of the American Chemical Society 138 (2016) pages 6805-6812 reports a bimetallic catalyst (Pd—Cu) supported on hydrotalcite in the 1-octanol Guerbet reaction.

US2023/037136 relates to a method for producing a Guerbet alcohol, comprising reacting a raw material alcohol having 8 or more and 22 or less carbon atoms, in the presence of a catalyst (A) containing a first component, a second component, and a third component below:
first component: copper,
second component: one kind selected from the group consisting of cobalt, nickel, molybdenum, and rhenium, and third component: at least one kind selected from the group consisting of titanium, iron, zinc, yttrium, zirconium, niobium, molybdenum, cerium, samarium, tantalum, tungsten, rhenium, and gold, and are different from the element selected as the second component.

WO201518793A1 (EP3030346A1) relates to a catalyst for condensing alcohols, which comprises partially or fully thermally decomposed support material in contact with iridium and/or ruthenium as promoter, wherein
a) the partially or fully decomposed support material comprises hydrotalcite-like compounds, preferably hydrotalcite, and/or precursor material of hydrotalcite-like compounds, preferably hydrotalcite precursor material, as starting material,
b) the proportion of Ru and/or Ir promoter is in the range 0.05-4% by weight, preferably 0.1-3.5% by weight and particularly preferably in the range 0.2-3.0% by weight and the average particle size of the promoter particles is <100 nm, preferably <75 nm, more preferably <50 nm and particularly preferably <20 nm,
c) the thermally decomposed support material has an Mg/Al ratio in the range 90/10-40/60, preferably from 90/10 to 70/30, where the ratio is based on the weight of the respective oxides; and a process for condensing alcohols by bringing an alcohol-comprising feed (gas) stream comprising an alcohol selected from the group consisting of C₁-C₅-alcohols into contact with the catalyst, wherein
(i) the process temperature is in the range from 200 to 450° C., preferably 250° C.-400° C.,
(ii) the process pressure is in the range 0.05-60 bar, more preferably 0.1-40 bar, particularly preferably 0.5-10 bar, even more preferably in the range 1-5 bar,
(iii) the alcohol content of the feed (gas) stream is in the range 0.5-90% by volume, preferably in the range 0.5-70% by volume and more preferably in the range 0.5-50% by volume,
(iv) the feed (gas) stream has a GHSV in the range 500-5000 h⁻¹, preferably in the range 1000-4000 h⁻¹, particularly preferably in the range 1000-2500 h⁻¹.

It is an object of the invention to provide an improved process for the manufacturing of polyisobutene from isobutene obtained from isobutanol obtained from condensing methanol and ethanol to iso-butanol in the gas phase.

In particular it is desirable to provide at least partially renewable starting materials for such a process since there is a need for a replacement of fossil starting materials in the chemical industry.

### CATALYST OF THE INVENTION

The objects mentioned here are achieved by discovery of a catalyst for condensing alcohols, especially a mixture of methanol and ethanol, which comprises (partially or fully thermally decomposed) support material in contact with copper as promoter, wherein
a) the (partially or fully decomposed) support material comprises hydrotalcite-like compounds, preferably hydrotalcite, and/or precursor material of hydrotalcite-like compounds, preferably hydrotalcite precursor material, as starting material,
b) the proportion of copper promoter is in the range 0.05 to 20% by weight, preferably 0.05 to 10% by weight and particularly preferably in the range of from 0.05 to 5.0% by weight,
c) the (thermally decomposed) support material has an Mg/Al ratio in the range 90/10-40/60, preferably from 90/10 to 70/30, where the ratio is based on the weight of the respective oxides, wherein the catalyst may optionally comprise at least one further promoter element from the group consisting of Pt, Rh, Ru, Pd, Co, Ni, Pd, Cu, Ag and Au and the content of further promoter is in the range 0.01 to 2% by weight, preferably 0.01 to 1% by weight, with the proviso that the amount of copper promoter is greater than the amount of further promoter. The support material and the promotor(s) add up to 100% by weight.

The copper comprising promoter particles are embedded in the matrix of the partially or fully thermally decomposed support material and are highly disperse, which has a favorable effect on the properties of the material and thus represents an important aspect of the invention. The catalysts of the invention described here have a high sintering resistance while the process of the invention is carried out.

In particular, copper in the combination according to the invention has been found to be a very particularly suitable promoter element, which was not to be expected in this form. Thus, a particularly suitable catalyst which displays totally extraordinary performance properties in the process of the invention has been able to be found on the basis of copper. The catalyst of the invention is thus superior to the catalysts and processes known from the prior art.

It is also worth mentioning that the content of copper-comprising promoter is very low and very good performance properties in the process of the invention for condensing alcohols are nevertheless achieved using the catalyst of the invention.

In addition, the catalyst of the invention can comprise at least one further promoter element. The further promoter elements comprise elements from the group consisting of Pt, Rh, Ru, Pd, Co, Ni, Pd, Cu, Ag and Au, with the content of further promoter elements or of further promoter element preferably being in the range 0.01 to 1% by weight, more preferably 0.01 to 0.5% by weight. The support material and the promotor(s) add up to 100% by weight.

In a preferred embodiment the catalyst comprises at least one further promoter element from the group consisting of Ru and Ir and the content of further promoter is in the range 0.01 to 2% by weight, preferably 0.01 to 1% by weight. In said embodiment the proportion of copper promoter is in the range 0.05 to 5.0%, especially 0.05 to 2.0% by weight and Ir, or Ru are preferably present in an amount of 0.05 to 0.15% by weight, more preferably about 0.1% by weight.

In a more preferred embodiment the proportion of copper promoter is in the range 0.05 to 5.0% by weight, especially 0.05 to 2.0% by weight and no further promoter element is present.

Hydrotalcite and/or Hydrotalcite Precursor Materials or Hydrotalcite-Like Compounds as Starting Material(s)

The term “hydrotalcite-like compound” is used as a generic term encompassing all compounds having the same basic structure as hydrotalcite as such. Hydrotalcite has the formula Mg₄Al₂(OH)₁₂CO₃·4H₂O.

The term “precursor material of hydrotalcite-like compound” is used to mean starting materials used for the production of hydrotalcite-like compounds”.

Hydrotalcite-like compounds are mixed hydroxides of divalent and trivalent metals, which are made up of polycations and have a layer structure. Hydrotalcite-like compounds are also referred to in the literature as anionic clays, layered double hydroxides (=LDHs), Feitknecht compounds or double layer structures. When starting from the parent compound hydrotalcite of the formula Mg₄Al₂(OH)₁₂CO₃·4H₂O, Al³⁺ can be partially or completely replaced by trivalent metal cations of similar size such as, for example, Ga³⁺, Fe³⁺, Mn³⁺ and Cr³⁺ and independently Mg²⁺ can be replaced by divalent cations of similar size, such as, for example, Mg²⁺, Zn²⁺, Fe²⁺ and Mn²⁺.

In a preferred embodiment, the hydrotalcite-like compound consists of hydrotalcite. The hydrotalcites used for the process of the invention preferably comprise magnesium as divalent metal and aluminum as trivalent metal. The metals of the hydrotalcites used preferably consist predominantly of magnesium and aluminum.

Another characteristic is that the hydrotalcite-like compounds and the promotor source are very intimately mixed.

Such mixing can, for example, be achieved by physical mixing of hydrotalcite-like and aluminum hydroxide-comprising powders, for example by powder mixing in suitable technical apparatuses such as mixers. Such intimate mixing processes are known to those skilled in the art. A further possibility is to mix the hydrotalcite-like powder and the aluminum hydroxide-comprising powder in suitable dispersion media. As dispersion media, it is possible to use, for example, water, alcohols such as methanol, ethanol, propanol, butanol, ethylene glycol and/or butanediol and ketones such as acetone or methyl ethyl ketone. It is also possible for the dispersion media to be present as mixtures and comprise surface-active agents such as surfactants. Examples of such surfactants are, inter alia, polyethylene glycols, mersolates, carboxylates, long-chain ammonium compounds such as CTAB.

The process of the invention is preferably carried out using hydrotalcites or hydrotalcite-like compounds having a particularly high purity. The process for preparing these hydrotalcite-like compounds as are particularly preferably used in the process of the invention is disclosed in DE19503522A1.

According to DE19503522A1 the hydrotalcites or hydrotalcite-like compounds are formed by hydrolysis of metal alkoxides by means of water and subsequent drying of the hydrolysis products which have separated out as precipitate. The metal alkoxides are formed by reaction of monovalent, divalent and/or trivalent alcohols with one or more divalent metals and/or one or more trivalent metals. The water used for the hydrolysis preferably comprises water-soluble anions selected from the group consisting of hydroxide anions, organic anions, in particular alkoxides, alkyl ether sulfates, aryl ether sulfates and/or glycol ether sulfates, and/or inorganic anions, in particular carbonate, hydrogencarbonate, chloride, nitrate, sulfate and/or polyoxometalate anions. Ammonium is preferably used as counterion.

Suitable starting materials are commercially available hydrotalcites which can be procured, for example, under the name Pural MG from Sasol. Otherwise, the hydrotalcites or the hydrotalcite precursor materials can also be prepared by a person skilled in the art, for example via the precipitation products of metal salts which are precipitated in the appropriate stoichiometric ratios and are converted by thermal treatment into the desired structures/desired structure.

The preferred starting material is a mixture of magnesium and aluminum, either as oxide mixture or as mixture of oxides and elemental metals.

Furthermore, it is also possible to synthesize hydrotalcite and/or hydrotalcite precursor material as starting material (the support precursor) from a finely divided aluminum oxide and aluminum hydroxide mixture, for example the commercially available material Disperal (Sasol), and a suitable magnesium source. As magnesium source, it is possible to use, for example, any water-soluble salt, with magnesium nitrates being particularly useful.

It has also been found that the magnesium cations or atoms can be partly replaced by other cations or atoms while maintaining the catalytic properties of the material. For example, magnesium can be replaced by other divalent alkaline earth metals, preferably calcium. In a further embodiment, it is possible to bring a mixture of calcium oxide and aluminum oxide into contact with a magnesium-comprising compound. Here, the calcium oxide and aluminum oxide should be present in a ratio of from 90/10 to 40/60, preferably from 90/10 to 70/30. As magnesium-comprising compound, it is possible to use, for example, any water-soluble salt, with magnesium nitrates being particularly useful. The ratio here is the weight ratio of the oxides.

The suitable starting material (i.e. support precursor) can also be synthesized by a precipitation process. For this purpose, an aqueous solution of the mixture of magnesium- and aluminum-comprising salts is prepared in such a way that magnesium and aluminum cations are present in the ratio described below. All water-soluble salts are suitable for the preparation of the salt mixture, with the nitrates having been found to be particularly useful. Furthermore, it is also possible to synthesize the starting material (or the support precursor) from a finely divided aluminum oxide and aluminum hydroxide mixture, for example the commercially available material Disperal (Sasol) and a suitable magnesium source. As magnesium source, it is possible to use, for example, any water-soluble salt, with magnesium nitrates being particularly useful.

The introduction of the promoter elements via suitable starting materials is also of importance. The background is that the homogeneity of the dispersion in the solution plays a critical role in the dispersion and distribution of the particles present in the catalyst of the invention. Suitable starting materials for precipitating or obtaining the Cu comprising promoters are: halides or other inorganic salts such as sulfates and nitrates, also acetates, acetylacetonates and oxalates, and also olefin complexes, complexes with pyridine or other amine ligands, carbonyl complexes and finally also complexes with phosphanes, phosphides and phosphates.

As examples of compounds which can be used for the catalyst synthesis, mention may be made of the following: Examples of Cu-comprising compounds: Cu(NO₃)₂, Cu₂SO₄, CuCl₂, Cu(OAc)₂ and Cu(O₂C₅H₇)₂. Examples of Ru-comprising compounds: Ru(NO)(NO₃)₃, Ru(NO₃)₃, RuCl₃, RuCl₃·xH₂O, Ru₃(CO)₁₂, Ru(OAc)₃, Ru(acac)₃, Ru(CO)₂(OAc)₂, RuCl₂(cod), [RuCl₂(C₆H₆)]₂, Ru(Cp)CI(PPh₃)₂, [Cp*RuCl]₄, RuHCl(PPh₃)₃, RuH₂(CO)(PPh₃)₃, RuCl₂(PPh₃)₃ and RuH₂(PPh₃)₄. Examples of iridium-comprising compounds: IrCl₃, IrCl₃·xH₂O, IrCl₄·xH₂O, IrO₂, Ir(OAc)₃, Ir(acac)₃, Ir(cod)(acac), IrH(CO)(PPh₃)₃, [Cp*IrCl₂]₂, [IrCl(cod)]₂ and Ir₄(CO)₁₂.

### Details of the Production of the Catalyst of the Invention

The invention also provides a catalyst for condensing alcohols, which comprises (partially or fully thermally decomposed) support material in contact with Cu as promoter, where the catalyst of the invention can be produced by the following steps:
d) support material comprising a hydrotalcite-like compound, preferably hydrotalcite-comprising support material, and/or precursor material of a hydrotalcite-like compound, preferably hydrotalcite precursor material, is brought into contact with a promoter source,
e) an intimate mixture of support material comprising a hydrotalcite-like compound, preferably hydrotalcite-comprising support material, and/or precursor material of a hydrotalcite-like compound, preferably hydrotalcite precursor material, and the promoter source is produced,
f) the intimate mixture of support material comprising a hydrotalcite-like compound, preferably hydrotalcite-comprising support material, and/or precursor material of a hydrotalcite-like compound, preferably hydrotalcite precursor material, and the promoter source is treated thermally, with the thermal treatment comprising a calcination process at a temperature in the range 200-1000° C., preferably 200-900° C. and particularly preferably 200-850° C.

The catalyst of the invention is based on a partially or fully thermally decomposed support material, where the thermal decomposition as per step c) of the production process is based on a calcination treatment. Also of central importance here is that the contacting with the promoter source as per step a) and the intimate mixing of the promoter source with the precursor material of the support material as per step b) always has to be carried out before the calcination treatment in step c).

In a preferred embodiment, the production process can comprise a multistage calcination process with a treatment at a first temperature level and a treatment at a second temperature level.

### Shaping and Molding of the Catalyst

The shaping step may be, for example, tableting, extrusion, spray drying, granulation or similar processes which are known to those skilled in the art. An extrusion or tableting process may be used for producing the catalyst.

The shaped bodies obtained here can be obtained in various sizes and shapes. For example, pellets have dimensions of 3 mm in length and 6 mm in diameter, 5 mm in length and 5 mm in diameter or 5 mm in length and 8 mm in diameter.

The compacting stage may also be carried out a number of times in succession in order to increase the efficiency. This compaction may be carried out on pulverulent starting materials using a roller compacter. Here, small amounts of water can also be added to the starting materials in order to convert the powder into a kneadable paste.

### Calcination Process

In a preferred embodiment of the production process, the calcination process comprises multistage heating of the catalyst at least two different temperature levels.

The use of preferred heating rates during heating-up of the samples is advantageous in order to avoid local exothermic combustion processes and associated sintering processes within the catalyst. In addition, it can be preferred that the samples are subjected to pre-drying in order to commence the calcination process with samples having a water content of not more than 50% by weight.

In a preferred calcination procedure, the sample impregnated with promoter species is, for example, calcined in a two-stage process in which the thermal treatment of the sample in the first calcination stage is carried out in the range from 200° C. to 300° C., preferably from 250° C. to 300° C., and the thermal treatment of the sample in the second calcination stage is carried out in the range from 350° C. to 1000° C., preferably from 400° C. to 900° C. and particularly preferably from 400° C. to 850° C. Furthermore, heating the impregnated sample at a controlled heating rate to the target temperatures can be preferred, with a preferred heating rate providing a temperature rise of 0.5-3.0 K/min, preferably 0.5-1.5 K/min. The calcination is preferably carried out under an air atmosphere, with the air more preferably being passed over the sample at a flow rate of 3-10 I/min, more preferably from 5 to 8 I/min. The amount of air passed through the furnace depends on the respective furnace volume.

The catalyst of the invention may have a high specific surface area, which can be determined by means of nitrogen sorption.

In a particularly preferred embodiment the present invention is directed to a catalyst for condensing alcohols, which comprises support material in contact with copper as promoter, wherein
a) the support material comprises hydrotalcite,
b) the proportion of copper promoter is in the range of from 0.05 to 2.0% by weight,
c) the support material has an Mg/Al ratio in the range of 90/10-40/60, preferably from 90/10 to 70/30, where the ratio is based on the weight of the respective oxides, wherein the catalyst comprises no further promoter element.

In another particularly preferred embodiment the present invention is directed to a catalyst for condensing alcohols, which comprises support material in contact with copper as promoter, wherein
a) the support material comprises hydrotalcite,
b) the proportion of copper promoter is in the range of from 0.05 to 5.0% by weight, especially 0.05 to 2.0% by weight,
c) the support material has an Mg/Al ratio in the range of 90/10-40/60, preferably from 90/10 to 70/30, where the ratio is based on the weight of the respective oxides, wherein the catalyst comprises at least one further promoter element from the group consisting of Ru and Ir and the content of further promoter is in the range of from 0.01 to 1.0% by weight, preferably 0.05 to 0.15% by weight, with the proviso that the amount of copper promoter is greater than the amount of further promoter.

The catalyst for condensing alcohols according to the particularly preferred embodiments is obtainable by a process comprising the following steps:
d) support material comprising hydrotalcite and/or precursor material of a hydrotalcite precursor material is brought into contact with a promoter source,
e) an intimate mixture of support material and the promoter source is produced,
f) the intimate mixture of support material and the promoter source is treated thermally, with the thermal treatment comprising a calcination process at a temperature in the range 200-1000° C., preferably 200-900° C. and particularly preferably 200-850° C.

The calcination process comprises preferably heating the catalyst at at least two different temperature levels.

The catalyst is preferably subjected to an activation treatment at 350 to 450° C. under 15 to 25 vol % H2 in inert gas for 1 to 30 h

The catalyst is preferably subjected to a conditioning treatment at 200 to 300° C. under the flow of 0.5 to 5 vol % ethanol for 12 to 48 h.

The catalyst is preferably subjected to an equilibration treatment under the above-mentioned conditions over 50 to 200 h time on stream.

The catalyst for condensing alcohols according to the particularly preferred embodiments is advantageously used in the process for manufacturing of polyisobutene as described herein.

### Process for Condensing Different Alcoholic Components

A preferred embodiment of the process of the invention relates to the condensation of different alcoholic components. Processes for condensing alcohols from feed gas streams which comprise a plurality of different alcoholic components.

In this embodiment of the process for condensing alcohols in conjunction with the catalyst of the invention,
(i) the process temperature is implemented in the range from 200 to 450° C., preferably 250° C. to 400° C.,
(ii) the process pressure is in the range 0.05 to 60 bar, more preferably 0.1 to 40 bar, particularly preferably 5 to 9 bar,
(iii) the alcohol content of the feed (gas) stream is in the range 0.5 to 90% by volume, preferably in the range 0.5 to 70% by volume and more preferably in the range 0.5 to 50% by volume,
(iv) the feed (gas) stream has a GHSV in the range 500-5000 h⁻¹, preferably in the range 1000-4000 h⁻¹.

The GHSV (gas hourly space velocity) is the quotient of gas volume flow and catalyst volume.

In a further and preferred embodiment of the process of the invention, the alcohol-containing feed gas stream comprises at least two different alcoholic components from the group consisting of the C₁-C₅alcohols. The first of the at least two components is methanol (i.e. component 1), and the second of the at least two components (i.e. component 2) is a component from the group consisting of C₂-C₅alcohols. The second of the at least two components is preferably a component from the group consisting of C₂-C₄alcohols. Particular preference is given to the second of the at least two components being a C₂alcohol or a C₃alcohol. Especially the second of the at least two components (i.e. component 2) is ethanol.

Accordingly the present invention relates to a process for condensing alcohols by bringing an alcohol-comprising feed gas stream comprising an alcoholic component selected from the group consisting of C₁-C₅alcohols, especially at least two different alcoholic components from the group consisting of C₁-C₅alcohols, wherein very especially the first of the at least two components is methanol (i.e. component 1), and the second of the at least two components (i.e. component 2) is a component selected from the group consisting of C₂-C₅alcohols, into contact with the above-mentioned catalyst, wherein
(i) the process temperature is in the range from 200 to 450° C., preferably 250° C. to 400° C.,
(ii) the process pressure is in the range 0.05 to 60 bar, more preferably 0.1 to 40 bar, particularly preferably 5 to 9 bar,
(iii) the alcohol content of the feed (gas) stream is in the range 0.5 to 90% by volume, preferably in the range 0.5 to 70% by volume and more preferably in the range 0.5 to 50% by volume, and
(iv) the feed (gas) stream has a GHSV in the range 500-5000 h⁻¹, preferably in the range 1000-4000 h⁻¹.

When the process of the invention is carried out using the at least two different components, the molar proportion of methanol is preferably higher than the molar proportion of the at least second component from the group consisting of C₂-C₅alcohols. The molar ratio of methanol to component 2 (i.e. n methanol/n comp. 2) is preferably in the range from 5:1 to 50:1, more preferably in the range from 10:1 to 40:1 and in particular in the range from 15:1 to 20:1.

The C₁-C₅alcohols used in the process of the present invention can alternatively be renewable raw materials. For instance, bioethanol or alcohols derived from fusel oil may be used.

In an embodiment, the stream which is obtained from the fusel oil contains the C₅-Cₑ primary alcohol has a pMC greater than 90 when measured by a method as described in the ASTM norm D6866 (the current version is D6866-22), which defines the concept of “percent Modern Carbon” or pMC. Preferably the pMC is greater than 91, preferably greater than 93, preferably greater than 95, preferably greater than 96, preferably greater than 97, more preferably greater than 98, even more preferably greater than 99.

In an embodiment, the verification that a feedstock was derived from renewable raw materials is possible according to ASTM D6866 via ¹⁴C for example. A feedstock shall be regarded as “derived from renewable raw materials” for the purposes of this invention when the carbon-14 (C-14) presence therein corresponds substantially (to within not more than 6%) to the ASTM D6866 content of C-14 in atmospheric CO₂.

The C-14 content of a material may be determined by determining the decays of C-14 in this material by liquid scintillation. Such raw materials shall preferably be regarded as derived from renewable raw materials when they have a C-14 content displaying a radioactive decay of not less than 1.5 dpm/gC (decays per minute per gram of carbon), preferably 2 dpm/gC, more preferably 2.5 dpm/gC and yet more preferably 5 dpm/gC.

“Renewably-based” or “renewable” denote that the carbon content of a biofuel precursor and subsequent products is from a “new carbon” source as measured by ASTM test method D 6866-05, “Determining the Biobased Content of Natural Range Materials Using Radiocarbon and Isotope Ratio Mass Spectrometry Analysis”, incorporated herein by reference in its entirety. This test method measures the ¹⁴C/¹²C isotope ratio in a sample and compares it to the ¹⁴C/¹²C isotope ratio in a standard 100% biobased material to give percent biobased content of the sample.

“Biobased materials” are organic materials in which the carbon comes from recently (on a human time scale) fixated CO₂ present in the atmosphere using sunlight energy (photosynthesis). On land, this CO₂ is captured or fixated by plant life (e.g., agricultural crops or forestry materials). In the oceans, the CO₂ is captured or fixated by photosynthesizing bacteria or phytoplankton. For example, a biobased material has a ¹⁴C/¹²C isotope ratio greater than 0. Contrarily, a fossil-based material, has a ¹⁴C/¹²C isotope ratio of about 0. The term “renewable” with regard to compounds such as alcohols or hydrocarbons (linear or cyclic alkanes/alkenes/alkynes, aromatic, etc.) refers to compounds prepared from biomass using thermochemical methods (e.g., Fischer-Tropsch catalysts), biocatalysts (e.g., fermentation), or other processes, for example as described herein.

A small amount of the carbon atoms of the carbon dioxide in the atmosphere is the radioactive isotope ¹⁴C. This ¹⁴C carbon dioxide is created when atmospheric nitrogen is struck by a cosmic ray generated neutron, causing the nitrogen to lose a proton and form carbon of atomic mass 14 (¹⁴C), which is then immediately oxidized to carbon dioxide. A small but measurable fraction of atmospheric carbon is present in the faun of 14CO₂.

Atmospheric carbon dioxide is processed by green plants to make organic molecules during the process known as photosynthesis. Virtually all forms of life on Earth depend on this green plant production of organic molecule to produce the chemical energy that facilitates growth and reproduction. Therefore, the ¹⁴C that forms in the atmosphere eventually becomes part of all life forms and their biological products, enriching biomass and organisms which feed on biomass with ¹⁴C. In contrast, carbon from fossil fuels does not have the signature 14C:12C ratio of renewable organic molecules derived from atmospheric carbon dioxide. Furthermore, renewable organic molecules that biodegrade to CO₂ do not contribute to global warming as there is no net increase of carbon emitted to the atmosphere.

Assessment of the renewably based carbon content of a material can be performed through standard test methods, e.g. using radiocarbon and isotope ratio mass spectrometry analysis. ASTM International (formally known as the American Society for Testing and Materials) has established a standard method for assessing the biobased content of materials. The ASTM method is designated ASTM-D6866.

The application of ASTM-D6866 to derive “biobased content” is built on the same concepts as radiocarbon dating, but without use of the age equations. The analysis is performed by deriving a ratio of the amount of radiocarbon (¹⁴C) in an unknown sample compared to that of a modern reference standard. This ratio is reported as a percentage with the units “pMC” (percent modern carbon). If the material being analyzed is a mixture of present day radiocarbon and fossil carbon (containing very low levels of radiocarbon), then the pMC value obtained correlates directly to the amount of biomass material present in the sample.

In a preferred embodiment the alcohols used in the present invention have pMC values of at least greater than 90, preferably pMC values of at least greater than 95, preferably pMC values of at least greater than 98, more preferably pMC values of at least greater than 99, more preferably pMC values of at least about 100, inclusive of all values and subranges there-between.

It is also conceivable to use one alcohol from renewable sources and one alcohol from fossil sources, e.g. ethanol or n-propanol from renewable sources and methanol from fossil sources or n-propanol from fossil sources and methanol from renewable sources.

It is also conceivable to use alcohols partly from fossil and partly from renewable sources.

However, it is preferred that both alcohols are at least partly obtained from renewable sources, even more preferred that both alcohols are essentially completely obtained from renewable sources.

In one embodiment of the present invention methanol is preferably at least partially derived from renewable raw materials, more preferably essentially completely derived from renewable raw materials.

For example, methanol may be obtained by hydrogenation of or genetically engineered microorganisms from carbon dioxide (CO₂) and/or carbon monoxide (CO) which is not produced from fossil sources specifically for the process. On the other hand, the use of carbon dioxide (CO₂) and/or carbon monoxide (CO) from exhaust gases or combustion gases, which would otherwise be released into the atmosphere without their use in any process, is advantageous. Preferably, the carbon dioxide (CO₂) and/or carbon monoxide (CO) used in the process comes from renewable raw materials, for example from combustion processes or fermentation. It is also advantageous if carbon dioxide (CO₂) is separated from the air and used in the process (carbon capture and utilisation (CCU)). In this case, the product obtained from the process represent a carbon sink.

In case of hydrogenation of CO₂ and/or CO to methanol it is preferred to used hydrogen which exhibits a molar share of deuterium ≤100 ppm, preferably in the range of from 10 to ≤95 ppm, more preferably in the range of from 10 to ≤90 ppm, most preferably in the range of from 10 to ≤80 ppm, based on the total hydrogen content. Very preferably such hydrogen is obtained by electrolysis based on electrical power generated at least in part from non-fossil energy.

In one embodiment of the present invention ethanol is preferably at least partially derived from renewable raw materials, more preferably essentially completely derived from renewable raw materials.

In one embodiment of the present invention n-propanol is preferably at least partially derived from renewable raw materials, more preferably essentially completely derived from renewable raw materials.

Ethanol or n-propanol are obtainable from fermentation of at least one carbohydrate substrate in the presence of microorganisms or enzymes.

Preferred are carbohydrate substrates, particularly preferred are sugars as monomeric form, and as biopolymeric form starch, fibres, lignin, cellulose and hemicellulose. Sugars are preferably selected from the group consisting of arabinose, fructose, galactose, glucose, lactose, mannose, maltose, sucrose and xylose. The carbohydrates may preferably be biopolymers based on one or more of these sugars, preferably starch, lignin, cellulose and hemicellulose.

The microorganism may be bacteria, fungi or yeasts, which may be genetically modified.

Fusel oil is well known in the art and comprises a mixture of light alcohols, fatty esters, terpenes and furfural. The alcohols comprised in fusel oil are mainly propanol, butanol, amyl alcohol, isoamyl alcohols and hexanol and optionally heavier linear alcohols such as C₇ or Cₛ alcohols.

Fusel oils, occasionally referred to as “amyl oils” or “fusels”, have compositions which vary depending on their origin (potato, beet, wheat, barley, etc. musts).

Fusel oil is a mixture of 5% to 20% of water, 60% to 95% of alcohols mainly consisting of linear or branched alkanols containing from 2 to 5 carbon atoms of impurities including but not limiting to furfurals, ethers, fatty acids, etc. which, may be up to 15%.

In an embodiment the composition of fusel oil is as follows:
Ethanol 5 to 40%,
1-Propanol 1 to 8%,
2-Propanol 0 to 1%,
2-Methylpropanol 5 to 15%,
1-Butanol-0 to 1%,
2-Methyl 1-butanol 10 to 30%,
3-Methyl 1-butanol (isoamyl alcohol)₂₅ to 70%,
   the combination of alkanols representing 100%.

In addition to the alcoholic components, the feed gas stream comprises a carrier gas. The carrier gas is inert gas (for example nitrogen) or reactive gas (for example hydrogen). In a preferred embodiment, the feed gas comprises reactive gas, with the reactive gas being able to be present together with inert gas.

In a preferred embodiment the hydrogen used as reactive gas exhibits a molar share of deuterium ≤100 ppm, preferably in the range of from 10 to 95 ppm, more preferably in the range of from 10 to ≤90 ppm, most preferably in the range of from 10 to 80 ppm, based on the total hydrogen content. Very preferably such hydrogen is obtained by electrolysis based on electrical power generated at least in part from non-fossil energy.

In a further embodiment, the process of the invention relates to the condensation of methanol and n-propanol to produce iso-butanol. The proportion of methanol in the feed gas stream is preferably from five to ten times higher than the proportion of n-propanol.

In a particularly preferred embodiment, the process of the invention relates to the condensation of methanol and ethanol to produce iso-butanol. The embodiment relates to a process in which one ethanol molecule is reacted with two methanol molecules. The proportion of methanol in the feed gas stream is preferably from five to twenty times, more preferably from fifteen to twenty times higher than the proportion of ethanol.

In the various embodiments of the process of the invention, it is important that the feed gas stream is brought into contact under the reaction condition specified in the disclosure with the catalyst of the invention, which is disclosed in more detail in the present description and the claims, and the alcoholic components are reacted.

As regards the ratio of alcohol-containing components to reactive gas, preference is given to this being in the range from 40:2.5 to 20:10, preferably from 20:2.5 to 20:20. Further preference is given to an embodiment of the process of the invention in which hydrogen is used as reactive gas.

In a further embodiment of the process, the ratio of alcohol to reactive gas is preferably in the range from 100:1 to 1:10. It is known to those skilled in the art that the amount of reactive gas used also depends on the respective process parameters and the information given here is not intended to constitute restrictions. In particular, optimization of the process parameters can also lead to the preferred amount of reactive gas used depending on the respective alcohol components and the reaction conditions.

On the reaction of EtOH and MeOH a reaction temperature of 325° C. using a feed comprising the EtOH and MeOH in a molar ratio of 1:20 in the presence of 20% by volume of H₂ at GHSV 1000 h⁻ at 7 bar reaction pressure results in high conversion, the good yields and selectivities to the target compound, preferably iso-butanol. In said embodiment the proportion of copper promoter in the catalyst is in the range 0.05 to 10% by weight, especially 0.05 to 5.0% by weight, such as, for example, 0.1 or 1.0% by weight and no further promoter element is present.

When copper catalysts are equilibrated under mixed Guerbet conditions (temperature of 325° C. using a feed comprising the EtOH and MeOH in a molar ratio of 1:20 in the presence of 20% by volume of H₂ at 7 bar reaction pressure) over 100 h time on stream, the selectivity of Cu catalysts can be changed from CO/CO₂ to Guerbet intermediates such as C₃/C₄ aldehydes and C₃/C₄ alcohols. This equilibration phase may also lead to the complete disappearance of CO₂ from the product spectrum over time on stream.

For example, a catalyst containing 0.1% by weight copper was more selective to C₄/C₃ products at the beginning and a catalyst containing 1.0% by weight copper produced more CO/CO₂, as with time on stream, the catalyst containing 1.0% by weight copper showed improved selectivity almost the same as the catalyst containing 0.1% by weight copper and also good conversion of EtOH and MeOH.

The high performance of the process of the invention in respect of the high conversion, the good yields and selectivities to the target compound, preferably iso-butanol, contribute, in particular, to an economical process having a high efficiency.

With regard to yields and selectivities, it should also be mentioned that the formation of by-products is not ruled out in the process of the invention for condensing alcohols. The by-products are formed in a much lower proportion than the main product or products. In the case of the by-products, a distinction has to be made between desirable by-products and undesirable by-products.

Products which are desirable in the reaction of ethanol are those products which can easily be converted into isobutanol, e.g. isobutanal and crotonaldehyde. In addition, products which can be converted into products of value by after-treatment, e.g. acetaldehyde, also count as desirable by-products. Undesirable products are, in particular, gases such as CO, propane and methane which cannot be converted further under the present reaction conditions. In addition, high-boiling compounds without functional groups, which can be formed by uncontrolled further reaction of dimerization products, are undesirable. The obtained isobutanol is separated and purified by methods, such as, for example, distillation.

In a further preferred embodiment, after the process of the invention has been carried out, the catalyst is treated by a regeneration process, which contributes to the catalyst regaining at least a large proportion of its initial activity.

With regard to the catalyst of the invention for condensing alcohols, it may be said that the catalyst precursor material is preferably free of ethylenediaminetetraacetic acid or anions of ethylenediaminetetraacetic acid.

Preference is additionally given to the intimate mixture being produced from support material comprising a hydrotalcite-like compound, preferably hydrotalcite-comprising support material, and/or precursor material of a hydrotalcite-like compound, preferably hydrotalcite precursor material, and the promoter source, where the mixture preferably does not comprise any ethylenediaminetetraacetic acid or anions of ethylenediaminetetraacetic acid.

Additional aspects of the present invention relate to the use of the isobutanol, obtained according to the process described above, preferably from sources of renewable raw materials, as starting material in the synthesis of isobutene and its use in the polymerisaiton to polyisobutene.

### Subjecting Isobutanol to Dehydration to Obtain Isobutylene

Isobutanol is subjected to dehydration in the presence of a catalyst so as to obtain isobutylene. Reference is made to Jean-Luc Dubois et al., Catalysis Today 418 (2023) 114126.

Isobutylene can, for example, be produced according to the method described in EP4129963A1, comprising producing isobutylene from isobutanol using the catalyst described therein.

The catalyst according to EP4129963A1 contains at least one metal selected from Group 6 to Group 14 metal elements in Period 4 to Period 6 of the periodic table, which is preferably selected from Mn, Fe, Co, Ni, Cu, and Zn, in alumina which includes alumina consisting of one or more crystal phases of a monoclinic crystal phase, a tetragonal crystal phase, and a cubic crystal phase, such as, for example, alumina containing y-alumina having a tetragonal crystal phase as a main component, BET specific surface area: 243 m²/g, Na₂O content: less than 0.050% by mass, SiO₂ Content: 0.10% by mass. The content of the metal is preferably 0.025 mmol or more with respect to 1 g of the alumina.

Dehydration of isobutanol may be carried out in the liquid phase or in the gas phase. When the reaction is carried out in the gas phase, a type of a fixed bed, a fluidized bed, or the like can be used.

For example, by vaporizing a raw material with a vaporizer, it can be supplied to a reactor as a raw material gas. The conditions for vaporizing the raw material are not particularly limited, and for example, the temperature can be 108° C. or higher and 600° C. or lower, and the pressure can be 0.05 MPa or higher and 1 MPa or lower in terms of absolute pressure. In the raw material gas, the isobutanol concentration can be adjusted by diluting isobutanol with a diluent gas. The raw material gas may be a gas consisting only of isobutanol. The diluent gas may be any gas that does not affect the dehydration of isobutanol. Oxygen or hydrogen may be used as a diluent gas. The diluent gas included in the raw material gas may be a mixture of two or more diluent gases. Moisture may be included in the raw material gas. The isobutanol concentration in the raw material gas is preferably 5.0% by volume or more, particularly preferably 15.0% by volume or more, even still more preferably 25.0% by volume or more, and most preferably 45.0% by volume or more, with respect to the total volume of the raw material gas. The upper limit is not particularly limited, and it is 100% by volume or less. The reaction temperature in the dehydration of isobutanol is preferably 200° C. or higher, more preferably 220° C. or higher, still more preferably 240° C. or higher, particularly preferably 260° C. or higher, and most preferably 280° C. or higher. The reaction pressure in the dehydration of isobutanol is preferably 50 kPa or more in terms of absolute pressure and preferably 600 kPa or less.

Alternatively, the method for producing isobutylene described in CN106582603A may be used, which comprises subjecting isobutanol to a dehydration reaction in the presence of a modified alumina catalyst produced by a process comprising the steps of:
a) mixing an aluminum salt solution and an alkali liquor;
b) aging the mixture obtained in the step a) to obtain pseudo-boehmite gel;
c) mixing the pseudo-boehmite gel obtained in the step b) with amorphous silica-alumina, drying, extruding and molding to obtain a carrier;
d) impregnating the carrier obtained in step c) with caustic alkali solution and calcium salt solution, drying and roasting.

Alternatively, the method described in WO2022/226371 may be used, which is directed to a process for converting one or more C₁-C₅ linear or branched alcohols to one or more C₂-C₅ olefins, the process comprising: contacting an input stream comprising the one or more C₁-C₅ linear or branched alcohols with at least a first catalyst and a second catalyst in a single bed reactor to form an output stream comprising the one or more C₂-C₅ olefins, the single bed reactor being at a temperature from about 350° C. to about 750° C., a gauge pressure from 0 to about 30 bar, and a weight hourly space velocity (WHSV) from about 0.5 to about 5.0, wherein the first catalyst comprises a doped or undoped alumina catalyst including, in neutral or ionic form, one or more of zirconium (Zr), titanium (Ti), tungsten (W), or silicon (Si), to form a first mixture; and wherein the second catalyst comprises a doped or undoped zeolite catalyst.

Alternatively, the method for producing isobutylene described in U.S. Ser. No. 10/464,860 may be used, which comprises introducing a reaction gas containing isobutanol into a reactor, wherein the reaction gas further contains water in a content of 0.1 to 70 vol %; and dehydrating the isobutanol at a reaction pressure of 50 kPa or more and 750 kPa or less as an absolute pressure by using an alumina catalyst having a BET specific surface area in a range of 60 m²/g or more and 175 m²/g or less and having a content of SiO₂ of less than 1.0 mass %.

In a preferred embodiment of U.S. Ser. No. 10/464,860 a reaction gas containing isobutanol is introduced into a reactor, wherein the reaction gas further contains water in a content of 0.1 to 70 vol %; and the isobutanol is dehydrated at a concentration of isobutanol relative to all gaseous components entering a reaction zone to be supplied of 30 vol % or more and 85 vol % or less, a weight hourly space velocity (WHSV) of isobutanol of 0.175 h⁻¹ or more and 20 h⁻¹ or less, and a reaction pressure of 50 kPa or more and 750 kPa or less as an absolute pressure, by using an alumina catalyst of which 90 mass % or more has a particle size in a range of 700 μm or more and 10000 μm or less.

The isobutylene may be separated and purified from a reaction gas containing isobutylene and unreacted isobutanol by the method described, for example, in U.S. Ser. No. 10/550,052B2 comprising:
a step (1) of contacting the reaction gas containing the isobutylene and unreacted isobutanol with a first solvent to obtain a first gas containing the isobutylene and a recovered solution containing the unreacted isobutanol;
a step (2) of contacting the first gas with a second solvent selected from tert-butanol, a tert-butanol aqueous solution, and methyl tert-butyl ether to allow the second solvent to absorb the isobutylene contained in the first gas to obtain an absorption solution containing the isobutylene, and a step (3) of distilling the absorption solution to obtain separated and purified isobutylene.

### Purification Step (Optionally)

In an optional step the reaction mixture either on the stage of the iso-butanol-containing mixture or the iso-butene-containing mixture or both may be purified to yield an olefin-containing product stream which can be further employed in the polymerisation.

In a preferred embodiment, at least one purification step is undergone in the course of the process according to the present invention, preferably exactly one purification step, and even more preferably such purification takes place on stage of the iso-butene-containing mixture.

Such iso-butene-containing mixture usually is a mixture comprising isobutene as target product, optionally olefins other than the target product, e.g. propene, 1-butene, 2-butene or higher olefines, the starting alcohol or other alcohols, the aldehyde or carboxylic acid corresponding to the starting or condensation alcohols, and esters of the listed carboxylic acids and alcohols.

The purification step is preferably selected from the group consisting of
distillation,
extraction,
membrane filtration,
reverse osmosis, and
sorption to inorganic materials.

Preferably the purification is conducted by distillation and/or extraction, more preferably by distillation or rectification.

Purification is preferably carried out by distillation or rectification, optionally supported by stripping with an inert gas. This can also be done by increasing the temperature of the reaction mixture and/or lowering the pressure, preferably by a combination of these two measures.

Single-stage distillation can be done either from the reactor or by passing through a suitable aggregate, such as rotary evaporator, thin film evaporator, falling film evaporator, wiper blade evaporator, sambay evaporator, etc., and combinations thereof.

Rectification is preferably carried out by distillation columns placed above the reactor, such as bottom columns, which can be equipped with internals, valves, side extractors, etc. if desired. The distillation column or columns used can be realized in a well-known design (see e.g. Sattler, Thermische Trennverfahren, 2nd edition 1995, Weinheim, pp. 135ff; Perry's Chemical Engineers Handbook, 7th ed. 1997, New York, Section 13). The distillation columns used may contain separating fixtures, such as separating floors, e.g. perforated bottoms, bell bottoms or valve bottoms, orderly packings, e.g. sheet or fabric packings, or irregular pouring of fillers. As a rule, up to 20, preferably up to 10 theoretical plates are sufficient.

An extraction is preferably carried out using polar solvents in order to separate the polar compounds in the reaction mixture from the hydrocarbons.

Suitable extractants may e.g. be n-butanol, iso-butanol, sek-butanol, 2-ethylhexanol, 2-propylheptanol, cyclopentanol, cyclohexanol, the C₅-C₈ primary alcohol serving as starting material, C₂-C₁₀-carboxylic acid C₁-C₁₀-alkyl esters, phthalic acid-C₁-C₁₀-alkyl esters, ketones, such as acetone, ethyl methyl ketone or diethyl ketone.

It is also possible to use hydrocarbons as extractants in order to separate the hydrocarbons in the reaction mixture from the polar compounds.

Such hydrocarbons as extractants are e.g. halogen-free aliphatic or cycloaliphatic, preferably aliphatic hydrocarbons, especially hexane, pentane, heptane, cyclohexane, cyclopentane, and mixtures comprising them. In a preferred embodiment the extractant is the same hydrocarbon which is also used as solvent in the polymerisation (see below).

In terms of process technology, all known extraction and washing processes and apparatus can be used for washing or extraction in purification step, e.g. those described in Ullmann's Encyclopedia of Industrial Chemistry, 6th ed, 1999 Electronic Release, Chapter: Liquid-Liquid Extraction-Apparatus. For example, these can be single- or multi-stage, preferably single-stage extractions, as well as those in direct or counter-current mode, preferably counter-current operation.

Preferably, sieve bottom or packed or packing columns, stirring tanks or mixer-settler devices, as well as pulsed columns or those with rotating internals are used.

Membrane filtration and reverse osmosis are known to the person skilled in the art and do not need to be explained further.

Sorption can occur on inorganic materials, such as silica gel, silicates, alumina, zeolites, diatomaceous earth, mixed aluminium/silicon oxides, as well as calcium carbonates and oxides, or on activated or charcoal.

### Polymerisation

In the polymerisation the isobutene obtained in the preceding steps, preferably in purified form, is polymerised in the presence of at least one Lewis Acid and in the presence of at least one initiator.

The isobutene obtained in the preceding steps may optionally be mixed with isobutene from other sources, preferably fossil sources. Hence, it is possible to obtain polyisobutene with a certain content of monomers from renewable as well as fossil sources.

In a preferred embodiment, the olefin utilised in the polymerisation predominantly originates from the condensation and dehydration steps described above and exhibits a pMC greater than 90, preferably at least 95, more preferably at least 98%, even more preferably at least 99%, or even 100%, when measured by a method as described in the ASTM norm D6866, preferably to an extent of at least 66%, more preferably to an extent of at least 75%, even more preferably to an extent of more than 85%, especially to at least 90%, and even completely from the condensation and dehydration steps.

In case the isobutene completely originates from renewable sources the resulting polyisobutene exhibits a pMC of at least 90%, preferably at least 95%, more preferably at least 98%, even more preferably at least 99%, or even 100%, when measured by a method as described in the ASTM norm D6866.

It is an advantage of polyisobutene obtained from this preferred embodiment that it is especially suitable as food grade or polyisobutene or for medical applications, e.g. for chewing gums, in adhesive compositions approved for food contact or in plasters.

It is a further advantage that the at least partial use of starting alcohols from renewable materials rather than fossil materials is advantageous for the carbon footprint of the product finally obtained.

In another embodiment of the present invention isobutene from the condensation and dehydration originates from from renewable sources may be admixed to isobutene from fossil sources to an extent of of at least 1%, preferably at least 2%, more preferably at least 5%, even more preferably at least 10%, and especially at least 25%. Higher contents are possible depending on the availability of the alcohols from renewable sources fed into the condensation step.

For the use of fossil isobutene or of an isobutene-comprising monomer mixture as the monomer to be polymerized, suitable isobutene sources are both pure isobutene and isobutenic C4 hydrocarbon streams, for example C4 raffinates, especially “raffinate 1”, C4 cuts from isobutane dehydrogenation, C4 cuts from steam crackers and from FCC crackers (fluid catalyzed cracking), provided that they have been substantially freed of 1,3-butadiene present therein. A C4 hydrocarbon stream from an FCC refinery unit is also known as “b/b” stream. Further suitable isobutenic C4 hydrocarbon streams are, for example, the product stream of a propylene-isobutane cooxidation or the product stream from a metathesis unit, which are generally used after customary purification and/or concentration. Suitable C4 hydrocarbon streams generally comprise less than 500 ppm, preferably less than 200 ppm, of butadiene. The presence of 1-butene and of cis- and trans-2-butene is substantially uncritical. Typically, the isobutene concentration in the C4 hydrocarbon streams mentioned is in the range from 40 to 60% by weight. For instance, raffinate 1 generally consists essentially of 30 to 50% by weight of isobutene, 10 to 50% by weight of 1-butene, 10 to 40% by weight of cis- and trans-2-butene, and 2 to 35% by weight of butanes; in the polymerization process according to the invention, the unbranched butenes in the raffinate 1 generally behave virtually inertly, and only the isobutene is polymerized.

In a preferred embodiment, the fossil monomer source used for the polymerization is a technical C4 hydrocarbon stream with an isobutene content of 1 to 100% by weight, especially of 1 to 99% by weight, in particular of 1 to 90% by weight, more preferably of 30 to 60% by weight, especially a raffinate 1 stream, a b/b stream from an FCC refinery unit, a product stream from a propylene-isobutane cooxidation or a product stream from a metathesis unit.

Especially when a raffinate 1 stream is used as the isobutene source, the use of water as the sole initiator or as a further initiator has been found to be useful, in particular when polymerization is effected at temperatures of −20° C. to +30° C., especially of 0° C. to +20° C. At temperatures of −20° C. to +30° C., especially of 0° C. to +20° C., when a raffinate 1 stream is used as the isobutene source, it is, however, also possible to dispense with the use of an initiator.

The raw material of C₄ compounds is usually selected from the group consisting of
(a) a C₄ compound material in which an isobutene amount is adjusted to 50 to 75% by weight, obtained by adding high purity isobutene having the isobutene amount of 90 to 100% by weight to C₄ raffinate-1 which is a remainder after extracting 1,3-butadiene from a C₄ compound derived during a naphtha degrading process;
(b) a C₄ compound material in which an isobutene amount is adjusted to 50 to 75% by weight, obtained by adding high amount isobutene mixture having isobutene amount of 80 to 97% by weight, which is generated in an olefin conversion unit (OCU) process that produces propylene by the metathesis of ethylene and 2-butene, to C₄ raffinate-1 which is a remainder after extracting 1,3-butadiene from a C₄ compound derived during a naphtha degrading process;
(c) a C₄ compound material in which an isobutene amount is adjusted to 50 to 75% by weight, obtained by adding high purity isobutene having the isobutene amount of 90 to 100% by weight to butane-butene oil (B-B oil) derived from crude oil refining process;
(d) a C₄ compound material in which an isobutene amount is adjusted to 50 to 75% by weight, obtained by adding high amount isobutene mixture having the isobutene amount of 80 to 97% by weight, which is generated in an olefin conversion unit (OCU) process that produces propylene by the metathesis of ethylene and 2-butene, to butane-butene oil (B-B oil) derived from crude oil refining process;
(e) a C₄ compound material in which an isobutene amount is adjusted to 50 to 75% by weight, obtained by adding a dilute solvent to high purity isobutene having an isobutene amount of 90 to 100% by weight;
(f) a C₄ compound material in which an isobutene amount is adjusted to 50 to 75% by weight, obtained by adding a dilute solvent to high amount isobutene mixture having the isobutene amount of 80 to 97% by weight, which is generated in an olefin conversion unit (OCU) process that produces propylene by the metathesis of ethylene and 2-butene;
(g) a C₄ compound material in which an isobutene amount is adjusted to 50 to 75% by weight, obtained by adding high purity isobutene having the isobutene amount of 90 to 100% by weight to a mixture generated in dehydrogenation reaction that converts isobutane to isobutene; and
(h) a C₄ compound material in which an isobutene amount is adjusted to 50 to 75% by weight, obtained by adding high amount isobutene mixture having the isobutene amount of 80 to 97% by weight, which is generated in an olefin conversion unit (OCU) process that produces propylene by the metathesis of ethylene and 2-butene to a mixture generated in dehydrogenation reaction that converts isobutane to isobutene.

The isobutenic monomer mixture mentioned may comprise small amounts of contaminants such as water, carboxylic acids or mineral acids, without there being any critical yield or selectivity losses. It is appropriate to prevent enrichment of these impurities by removing such harmful substances from the isobutenic monomer mixture, for example by adsorption on solid adsorbents such as activated carbon, molecular sieves or ion exchangers.

It is also possible to convert monomer mixtures of isobutene or of the isobutenic hydrocarbon mixture with olefinically unsaturated monomers copolymerizable with isobutene. When monomer mixtures of isobutene are to be copolymerized with suitable comonomers, the monomer mixture preferably comprises at least 5% by weight, more preferably at least 10% by weight and especially at least 20% by weight of isobutene, and preferably at most 95% by weight, more preferably at most 90% by weight and especially at most 80% by weight of comonomers.

Useful copolymerizable monomers include: vinylaromatics such as styrene and a-methylstyrene, C1- to C4-alkylstyrenes such as 2-, 3- and 4-methylstyrene, and 4-tert-bu-tylstyrene, halostyrenes such as 2-, 3- or 4-chlorostyrene,

and isoolefins having 5 to 10 carbon atoms, such as 2-methylbutene-1, 2-methylpentene-1, 2-methylhexene-1, 2-ethylpentene-1, 2-ethylhexene-1 and 2-propylheptene-1. Further useful comonomers include olefins which have a silyl group, such as 1-trimethoxysilylethene, 1-(tri-methoxysilyl)propene, 1-(trimethoxysilyl)-2-methylpropene-2, 1-[tri(methoxyethoxy)-silyl]-ethene, 1-[tri(methoxyethoxy)silyl]propene, and 1-[tri(methoxyethoxy)silyl]-2-methylpro-pene-2. In addition—depending on the polymerization conditions—useful comonomers also include isoprene, 1-butene and cis- and trans-2-butene.

When the process according to the invention is to be used to prepare copolymers, the process can be configured so as to preferentially form random polymers or to preferentially form block copolymers. To prepare block copolymers, for example, the different monomers can be supplied successively to the polymerization reaction, in which case the second comonomer is especially not added until the first comonomer is already at least partly polymerized. In this manner, diblock, triblock and higher block copolymers are obtainable, which, according to the sequence of monomer addition, have a block of one or the other comonomer as a terminal block. In some cases, however, block copolymers also form when all comonomers are supplied to the polymerization reaction simultaneously, but one of them polymerizes significantly more rapidly than the other(s). This is the case especially when isobutene and a vinylaromatic compound, especially styrene, are copolymerized in the process according to the invention. This preferably forms block copolymers with a terminal polystyrene block. This is attributable to the fact that the vinylaromatic compound, especially styrene, polymerizes significantly more slowly than isobutene.

The polymerization can be effected either continuously or batchwise.

The process according to the invention is suitable either for performance at low temperatures, e.g. at −90° C. to 0° C., or at higher temperatures, i.e. at at least 0° C., e.g. at 0° C. to +30° C. or at 0° C. to +50° C. The polymerization in the process according to the invention is, however, preferably performed at relatively low temperatures, generally at −70° C. to −10° C., especially at −60° C. to −15° C.

When the polymerization in the process according to the invention is effected at or above the boiling temperature of the monomer or monomer mixture to be polymerized, it is preferably performed in pressure vessels, for example in autoclaves or in pressure reactors.

The polymerisation comprises polymerizing isobutene or an isobutene-comprising monomer mixture mixture which is at least partly obtained by a process comprising the condensation and dehydration steps in the presence of at least one Lewis Acid effective as a polymerization catalyst.

The Lewis Acid is preferably selected from the group consisting of aluminum trihalide, alkylaluminum halide, iron trihalide, a gallium trihalide, a titanium tetrahalide, a zinc dihalide, a tin dihalide, a tin tetrahalide, and a boron trihalide, more preferably selected from the group consisting of aluminum trihalide, alkylaluminum halide, titanium tetrahalide, and boron trihalide, even more preferably selected from the group consisting of aluminum trihalide, alkylaluminum halide, and boron trihalide, and especially the metal halide is aluminum trihalide or boron trihalide or even aluminum trichloride or boron trifluoride.

In a preferred embodiment, the Lewis Acid is deployed together with a donor forming a Lewis-Acid-donor complex, preferably selected from the group consisting of an aluminum trihalide-donor complex, an alkylaluminum halide-donor complex, an iron trihalide-donor complex, a gallium trihalide-donor complex, a titanium tetrahalide-donor complex, a zinc dihalide-donor complex, a tin dihalide-donor complex, a tin tetrahalide-donor complex, and a boron trihalide-donor complex, said complex comprising, as the donor, at least one organic compound (II) comprising at least one oxygen or nitrogen atom with at least one lone electron pair, preferably comprising at least one oxygen atom with at least one lone electron pair, very preferably selected from the group consisting of organic compounds with at least one ether function, organic compounds with at least one carboxylic ester function, organic compounds with at least one aldehyde function, organic compounds with at least one keto function, and organic compounds with at least one nitrogen containing heterocyclic ring.

A suitable aluminum trihalide is especially aluminum trifluoride, aluminum trichloride or aluminum tribromide, preferably aluminum trichloride.

A useful alkylaluminum halide is especially a mono(C₁- to C₄-alkyl)aluminum dihalide or a di(C₁- to C₄-alkyl)aluminum monohalide, for example methylaluminum dichloride, ethylaluminum dichloride, iso-butylaluminum dichloride, dimethylaluminum chloride or diethylaluminum chloride, diiso-butylaluminum chloride, preferably ethylaluminum dichloride, iso-butylaluminum dichloride, diethylaluminum chloride or diiso-butylaluminum chloride and very preferably ethylaluminum dichloride and iso-butylaluminum dichloride.

In the context of the present invention C₁ to C₄-alkyl is, for example, methyl, ethyl, propyl, isopropyl, n-butyl, sec-butyl or tert-butyl.

Especially suitable iron trihalides are iron trifluoride, iron trichloride or iron tribromide, preferably iron trichloride.

Especially suitable gallium trihalides are gallium trifluoride, gallium trichloride or gallium tribromide, preferably gallium trichloride.

Especially suitable titanium tetrahalides are titanium tetrafluorides, titanium tetrachlorides or titanium tetrabromides, preferably titanium tetrachlorides.

Especially suitable zinc dihalides are zinc difluorides, zinc dichlorides, or zinc dibromides, preferably zinc dichlorides.

Especially suitable tin dihalides are tin difluorides, tin dichlorides, or tin dibromides, preferably tin dichlorides.

Especially suitable tin tetrahalides are tin tetrafluorides, tin tetrachlorides, or tin tetrabromides, preferably tin tetrachlorides.

Especially suitable boron trihalides are boron trifluoride, boron trichloride, and boron tribromide, preferably boron trifluoride and boron trichloride, and more preferably boron trifluoride.

Among these Lewis Acids, aluminum trihalides, alkylaluminum halides, iron trihalides, titanium tetrahalides, and boron trihalides are preferred.

Very preferred are aluminum trihalides, alkylaluminum halides, iron trihalides, and boron trihalides, particularly preferred are boron trihalides and aluminum trihalides, especially boron trifluorides and aluminium trichloride.

In a preferred embodiment, isobutene or an isobutene-comprising monomer mixture is polymerized in the presence of an alkyl aluminum dichloride-donor complex or an dialkyl aluminum chloride-donor complex effective as a polymerization catalyst, very preferably in the presence of an aluminum trichloride-donor complex or an iron trichloride-donor complex.

In another preferred embodiment, isobutene or an isobutene-comprising monomer mixture is polymerized in the presence of a boron trifluoride-donor complex.

According to the invention the aluminum trihalide-donor complex or alkylaluminum halide-donor complex or the iron trihalide-donor complex or the gallium trihalide-donor complex or the titanium tetrahalide-donor complex or the zinc dihalide-donor complex or the tin dihalide-donor complex or the tin tetrahalide-donor complex or the boron trihalide-donor complex effective as a polymerization catalyst comprises a mixture of the respective metal halide with at least one organic compound (II) as the donor comprising at least one oxygen or nitrogen atom with at least one lone electron pair, preferably comprising at least one oxygen atom with at least one lone electron pair, very preferably selected from the group consisting of organic compounds with at least one ether function, organic compounds with at least one carboxylic ester function, organic compounds with at least one aldehyde function, organic compounds with at least one keto function, and organic compounds with at least one nitrogen containing heterocyclic ring

Compounds (II) comprise at least one oxygen and/or nitrogen atom with at least one lone electron pair, preferably at least one oxygen atom with at least one lone electron pair and very preferably are selected from the group consisting of organic compounds with at least one ether function, organic compounds with at least one carboxylic ester function, organic compounds with at least one aldehyde function, organic compounds with at least one keto function, and organic compounds with at least one nitrogen containing heterocyclic ring.

Solely oxygen containing compounds (II) are preferred over nitrogen-containing compounds (II).

Preferably compound (II) is selected from the group consisting of organic compounds with at least one ether function, organic compounds with at least one carboxylic ester function and organic compounds with at least one keto function, more preferably selected from the group consisting of organic compounds with at least one ether function and organic compounds with at least one carboxylic ester function, very preferably compounds (II) are organic compounds with at least one ether function, and especially organic compounds with exactly one ether function.

Compounds with at least one ether function are also understood to mean acetals and hemiacetals. The ether compound may comprise one or more ether functions, e.g. one, two, three, four or even more ether functions, preferably one or two ether functions and very preferably one ether function.

The mixture of donors may comprise one, two, three, four or even more different compounds (II), preferably compounds with at least one ether function, preferably one or two different compounds and very preferably one compound.

In a preferred embodiment of the present invention, an aluminum trihalide-donor complex or an alkylaluminum halide complex, or an iron trihalide-donor complex, or a gallium trihalide-donor complex or a titanium tetrahalide-donor complex or a zinc dihalide-donor complex or a tin dihalide-donor complex or the tin tetrahalide-donor complex or the boron trihalide-donor complex, very preferably an aluminum trihalide-donor complex or an iron trihalide-donor complex or a boron trihalide-donor complex and especially an aluminum trihalide-donor complex is used, which comprises, as the donor, a mixture of
at least one dihydrocarbyl ether the general formula R⁸—O—R⁹ in which the variables R⁸ and R⁹ are each independently C₁- to C₂₀-alkyl radicals, preferably C₁- to Cₛ alkyl radicals especially C₁- to C₄ alkyl radicals, C₁- to C₂₀-haloalkyl radicals, preferably C₁- to C₈ haloalkyl radicals especially C₁- to C₄ haloalkyl radicals, C₅- to C₈-cycloalkyl radicals, preferably C₅- to C₆-cycloalkyl radicals, C₆- to C₂₀-aryl radicals, especially C₆- to C₁₂ aryl radicals, C₆- to C₂₀-haloaryl radicals, especially C₆- to C₁₂ haloaryl radicals, or C₇- to C₂₀-arylalkyl radicals, especially C₇- to C₁₂-arylalkyl radicals. Preference is given to C₁- to C₄ alkyl radicals, C₁- to C₄ haloalkyl radicals, C₆- to C₁₂ aryl radicals, and C₇- to C₁₂-arylalkyl radicals.

A C₁- to C₈-alkyl radical is a linear or branched alkyl radical having 1 to 8 carbon atoms. Examples thereof are methyl, ethyl, n-propyl, isopropyl, n-butyl, 2-butyl, isobutyl, tert-butyl, pentyl, 1-methylbutyl, 2-methylbutyl, 3-methylbutyl, 2,2-dimethyl-propyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl and the constitutional isomers thereof, such as 2-ethylhexyl. Such C₁- to C₈-alkyl radicals may to a small extent also comprise heteroatoms such as oxygen, nitrogen or halogen atoms, for example chlorine, and/or aprotic functional groups, for example carboxyl ester groups, cyano groups or nitro groups.

A C₁- to C₂₀-alkyl radical is a linear or branched alkyl radical having 1 to 20 carbon atoms. Examples thereof are the abovementioned C₁- to C₈-alkyl radicals, and additionally n-nonyl, isononyl, n-decyl, 2-propylheptyl, n-undecyl, n-dodecyl, n-tridecyl, isotridecyl, n-tetradecyl, n-hexadecyl, n-octadecyl and n-eicosyl. Such C₁- to C₂₀-alkyl radicals may to a small extent also comprise heteroatoms such as oxygen, nitrogen or halogen atoms, for example chlorine, and/or aprotic functional groups, for example carboxyl ester groups, cyano groups or nitro groups.

A C₅- to C₈-cycloalkyl radical is a saturated cyclic radical which may comprise alkyl side chains. Examples thereof are cyclopentyl, 2- or 3-methylcyclopentyl, 2,3-, 2,4- or 2,5-dimethylcyclo-pentyl, cyclohexyl, 2-, 3- or 4-methylcyclohexyl, 2,3-, 2,4-, 2,5-, 2,6-, 3,4-, 3,5- or 3,6-dimethyl-cyclohexyl, cycloheptyl, 2-, 3- or 4-methylcycloheptyl, cyclooctyl, 2-, 3-, 4- or 5-methylcyclooctyl. Such C₅- to C₈-cycloalkyl radicals may to a small extent also comprise heteroatoms such as oxygen, nitrogen or halogen atoms, for example chlorine, and/or aprotic functional groups, for example carboxyl ester groups, cyano groups or nitro groups.

A C₆- to C₂₀-aryl radical or a C₆- to C₁₂-aryl radical is preferably optionally substituted phenyl, optionally substituted naphthyl, optionally substituted anthracenyl or optionally substituted phenanthrenyl. Such aryl radicals may be a 1 to 5 aprotic substituents or aprotic functional groups, for example C₁- to C₈-alkyl, C₁- to C₈-haloalkyl such as C₁- to C₈-chloroalkyl or C₁- to C₈-fluoroalkyl, halogens such as chlorine or fluorine, nitro, cyano or phenyl. Examples of such aryl radicals are phenyl, naphthyl, biphenyl, anthracenyl, phenanthrenyl, tolyl, nitrophenyl, chlorophenyl, dichlorophenyl, pentafluorophenyl, pentachlorophenyl, (trifluoromethyl)phenyl, bis(tri-fluoromethyl)phenyl, (trichloro)methylphenyl and bis(trichloromethyl)phenyl.

A C₇- to C₂₀-arylalkyl radical or a C₇- to C₁₂-arylalkyl radical is preferably optionally substituted C₁- to C₄-alkylphenyl such as benzyl, o-, m- or p-methylbenzyl, 1- or 2-phenyl-ethyl, 1-, 2- or 3-phenylpropyl or 1-, 2-, 3- or 4-phenylbutyl, optionally substituted C₁- to C₄-alkylnaphthyl such as naphthylmethyl, optionally substituted C₁- to C₄-alkylanthracenyl such as anthracenylmethyl, or optionally substituted C₁- to C₄-alkylphenanthrenyl such as phenanthrenylmethyl. Such arylalkyl radicals may bear 1 to 5 aprotic substituents or aprotic functional groups, especially on the aryl moiety, for example C₁- to C₈-alkyl, C₁- to C₈-halo-alkyl such as C₁- to C₈-chloroalkyl or C₁-to C₈-fluoroalkyl, halogen such as chlorine or fluorine, nitro or phenyl.

Haloalkyl and haloaryl mean preferably chloroalkyl or bromoalkyl and chloroaryl or bromo-aryl, very preferably chloroalkyl and chloroaryl. Especially preferred are w-haloalkyl radicals.

Preferred examples are chloromethyl, 1-chloroeth⁻¹-yl, 2-chloroeth⁻¹-yl, 2-chloroprop-1-yl, 2-chloroprop-2-yl, 3-chloroprop-1-yl, and 4-chlorobut-1-yl.

Preferred examples for chloroaryl are 2-chlorophenyl, 3-chlorophenyl, and 4-chlorophenyl.

The dihydrocarbyl ethers mentioned may be open-chain or cyclic, where the two variables R⁸ and R⁹ in the case of the cyclic ethers may join to form a ring, where such rings may also comprise two or three ether oxygen atoms.

Examples of such open-chain and cyclic dihydrocarbyl ethers are dimethyl ether, chloromethyl methyl ether, bis (chloromethyl) ether, diethyl ether, chloromethyl ethyl ether, 2-chloroethyl ethyl ether (CEE), bis(2-chloroethyl) ether (CE), di-n-propyl ether, diisopropyl ether, di-n-butyl ether, di-sec-butyl ether, diisobutyl ether, di-n-pentyl ether, di-n-hexyl ether, di-n-heptyl ether, di-n-octyl ether, di-(2-ethylhexyl) ether, methyl n-butyl ether, methyl sec-butyl ether, methyl isobutyl ether, methyl tert-butyl ether, ethyl n-butyl ether, ethyl sec-butyl ether, ethyl isobutyl ether, ethyl tert-butyl ether, n-propyl-n-butyl ether, n-propyl sec-butyl ether, n-propyl isobutyl ether, n-propyl tert-butyl ether, isopropyl n-butyl ether, isopropyl sec-butyl ether, isopropyl isobutyl ether, isopropyl tert-butyl ether, methyl n-hexyl ether, methyl n-octyl ether, methyl 2-ethylhexyl ether, ethyl n-hexyl ether, ethyl n-octyl ether, ethyl 2-ethylhexyl ether, n-butyl n-octyl ether, n-butyl 2-ethyl-hexyl ether, tetrahydrofuran, tetrahydropyran, 1,2-, 1,3- and 1,4-dioxane, dicyclohexyl ether, diphenyl ether, alkyl aryl ethers, such as anisole and phenetole, ditolyl ether, dixylyl ether and dibenzyl ether.

Furthermore, difunctional ethers such as dialkoxybenzenes, preferably dimethoxybenzenes, very preferably veratrol, and ethylene glycol dialkylethers, preferably ethylene glycol dimethylether and ethylene glycol diethylether, are preferred.

Among the dihydrocarbyl ethers mentioned, diethyl ether, 2-chloroethyl ethyl ether, diisopropyl ether, di-n-butyl ether and diphenyl ether have been found to be particularly advantageous as donors for the aluminum trihalide-donor complexes or the alkylaluminum halide complexes or the iron trihalide-donor complexes or the gallium trihalide-donor complex or the titanium tetrahalide-donor complex or the zinc dihalide-donor complex or the tin dihalide-donor complex or the tin tetrahalide-donor complex or the boron trihalide-donor complex, very preferably the aluminum trihalide-donor complexes or iron trihalide-donor complexes or boron trihalide-donor complex and especially the aluminum trihalide-donor complexes.

In a preferred embodiment dihydrocarbyl ethers with at least one secondary or tertiary dihydrocarbyl group are preferred over dihydrocarbyl groups with primary groups only. Ethers with primary dihydrocarbyl groups are those ethers in which both dihydrocarbyl groups are bound to the ether functional group with a primary carbon atom, whereas ethers with at least one secondary or tertary dihydrocarbyl group are those ethers in which at least one dihydrocarbyl group is bound to the ether functional group with a secondary or tertiary carbon atom.

For the sake of clarity, e.g. diisobutyl ether is deemed to be an ether with primary dihydrocarbyl groups, since the secondary carbon atom of the isobutyl group is not bound to the oxygen of the functional ether group but the hydrocarbyl group is bound via a primary carbon atom.

Preferred examples for ethers with primary dihydrocarbyl groups are diethyl ether, di-n-butyl ether, and di-n-propyl ether.

Preferred examples for ethers with at least one secondary or tertary dihydrocarbyl group are diisopropyl ether, methyl tert-butyl ether, ethyl tert-butyl ether, and anisole.

In addition, particularly advantageous dihydrocarbyl ethers as donors for the aluminum trihalide-donor complexes or the alkylaluminum halide complexes, have been found to be those in which the donor compound has a total carbon number of 3 to 16, preferably of 4 to 16, especially of 4 to 12, in particular of 4 to 8.

In another preferred embodiment halide-substituted ethers are preferred in combination with aluminum halide-donor complex or iron halide-donor complex or boron halide-donor complex.

Organic compounds with at least one carboxylic ester function are preferably hydrocarbyl carboxylates of the general formula R¹⁰—COOR¹¹ in which the variables R¹⁰ and R¹¹ are each independently C₁- to C₂₀-alkyl radicals, especially C₁- to Cₛ alkyl radicals, C₅- to C₈-cycloalkyl radicals, C₆- to C₂₀-aryl radicals, especially C₆- to C₁₂ aryl radicals, or C₇- to C₂₀-arylalkyl radicals, especially C₇- to C₁₂-arylalkyl radicals.

Examples of the hydrocarbyl carboxylates mentioned are methyl formate, ethyl formate, n-pro-pyl formate, isopropyl formate, n-butyl formate, sec-butyl formate, isobutyl formate, tert-butyl formate, methyl acetate, ethyl acetate, n-propyl acetate, isopropyl acetate, n-butyl acetate, sec-butyl acetate, isobutyl acetate, tert-butyl acetate, methyl propionate, ethyl propionate, n-propyl propionate, isopropyl propionate, n-butyl propionate, sec-butyl propionate, isobutyl propionate, tert-butyl propionate, methyl butyrate, ethyl butyrate, n-propyl butyrate, isopropyl butyrate, n-bu-tyl butyrate, sec-butyl butyrate, isobutyl butyrate, tert-butyl butyrate, methyl cyclohexanecarboxylate, ethyl cyclohexanecarboxylate, n-propyl cyclohexanecarboxylate, isopropyl cyclohexane-carboxylate, n-butyl cyclohexanecarbo-xylate, sec-butyl cyclohexanecarboxylate, isobutyl cyclohexanecarboxylate, tert-butyl cyclohexanecarboxylate, methyl benzoate, ethyl benzoate, n-pro-pyl benzoate, isopropyl benzoate, n-butyl benzoate, sec-butyl benzoate, isobutyl benzoate, tert-butyl benzoate, methyl phenylacetate, ethyl phenylacetate, n-propyl phenylacetate, isopropyl phenylacetate, n-butyl phenylacetate, sec-butyl phenylacetate, isobutyl phenylacetate and tert-butyl phenylacetate. Among the hydrocarbyl carboxylates mentioned, ethyl acetate has been found to be particularly advantageous as a donor for the complexes.

In addition, particularly advantageous hydrocarbyl carboxylates as donors, have been found to be those in which the donor compound has a total carbon number of 3 to 16, preferably of 4 to 16, especially of 4 to 12, in particular of 4 to 8, preference is given in particular to those having a total of 3 to 10 and especially 4 to 6 carbon atoms.

Organic compounds with at least one aldehyde function, preferably exactly one aldehyde function and organic compounds with at least one keto function, preferably exactly one keto function typically have from 1 to 20, preferably from 2 to 10 carbon atoms. Functional groups other than the carbonyl group are preferably absent.

Preferred organic compounds with at least one aldehyde function are those of formula R¹⁰—CHO, in which R¹⁰ has the above-mentioned meaning, very preferably are selected from the group consisting of formaldehyde, acetaldehyde, propionaldehyde, n-butyraldehyde, isobutyraldehyde, and benzaldehyde.

Preferred organic compounds with at least one keto function are those of formula R¹⁰—(C═O)—R¹¹, in which R¹⁰ and R¹¹ have the above-mentioned meaning, very preferably are selected from the group consisting of acetone, methyl ethyl ketone, diethyl ketone, methyl isobutyl ketone, cyclohexanone, acetophenone, and benzophenone. Greatest preference is given to acetone.

Organic compounds with at least one nitrogen containing heterocyclic ring are preferably saturated, partly unsaturated or unsaturated nitrogen-containing five-membered or six-membered heterocyclic rings which comprises one, two or three ring nitrogen atoms and may have one or two further ring heteroatoms from the group of oxygen and sulphur and/or hydrocarbyl radicals, especially C₁- to C₄-alkyl radicals and/or phenyl, and/or functional groups or heteroatoms as substituents, especially fluorine, chlorine, bromine, nitro and/or cyano, for example pyrrolidine, pyrrole, imidazole, 1,2,3- or 1,2,4-triazole, oxazole, thiazole, piperidine, pyrazane, pyrazole, pyridazine, pyrimidine, pyrazine, 1,2,3-, 1,2,4- or 1,2,5-triazine, 1,2,5-oxathiazine, 2H-1,3,5-thiadiazine or morpholine.

However, a very particularly suitable nitrogen-containing basic compound of this kind is pyridine or a derivative of pyridine (especially a mono-, di- or tri-C₁- to C₄-alkyl-substituted pyridine) such as 2-, 3-, or 4-methylpyridine (picolines), 2,3-, 2,4-, 2,5-, 2,6-, 3,4-, 3,5- or 3,6-dimethylpyridine (lutidines), 2,4,6-trimethylpyridine (collidine), 2-, 3, - or 4-tert-butylpyridine, 2-tert-butyl-6-methyl-pyridine, 2,4-, 2,5-, 2,6- or 3,5-di-tert-butylpyridine or else 2-, 3, - or 4-phenylpyridine.

The molar ratio of the donor compounds mentioned to the aluminum trihalide or to the alkylaluminum halide or to the iron trihalide or to the gallium trihalide or to the titanium tetrahalide or to the zinc dihalide or to the tin dihalide or to the tin tetrahalide or to the boron trihalide, in the donor complex generally varies within the range from 0.1:1 to 2.0:1, especially from 0.2:1 to 1.8:1, in particular 0.2:1 to 1.6:1; in most cases it is 0.4:1 to 1.5:1. However, it is also possible to work with a greater excess of the donor compounds, often up to a 10-fold and especially 3-fold molar excess; the excess amount of donor compounds then additionally acts as a solvent or diluent.

The molar ratio of the Lewis Acid mentioned, preferably the boron halide, aluminum trihalide or alkylaluminum halide mentioned to the isobutene monomer used in the case of homopolymerization of isobutene, or to the total amount of the polymerizable monomers used in the case of copolymerization of isobutene, based on each individual functional site of the Lewis Acid mentioned, preferably the boron halide, aluminum trihalide or alkylaluminum halide, is generally from 0.001:1 to 0.2:1, preferably 0.002:1 to 0.1:1, very preferably 0.003:1 to 0.08:1, especially 0.005:1 to 0.05:1, and in particular 0.007:1 to 0.03:1.

Typically, the Lewis Acid-donor complex, preferably the boron trihalide-donor complex, aluminum trihalide-donor complex or the alkylaluminum halide complex, especially the alkyl aluminum dichloride-donor complex or dialkyl aluminum chloride-donor complex, is prepared separately prior to the polymerization from the respective metal halides, preferably the aluminum trihalide or the alkylaluminum halide, especially from anhydrous alkyl aluminum dichloride or an dialkyl aluminum chloride, and the donor compound, and is then—usually dissolved in an inert solvent such as a halogenated hydrocarbon, for example dichlorome-thane, or more preferably in unhalogenated hydrocarbons—added to the polymerization medium. However, in a less preferred embodiment the complex can also be prepared in situ prior to the polymerization.

In case the polymerization catalyst is not fully soluble in the solvent used it may be advantageous to disperse the polymerization catalyst in the inert solvent, e.g. by vigorously stirring the dispersion. The dispersing can be effected in any apparatus suitable for dispersing. Shaking apparatuses such as for example from Skandex may be mentioned by way of example or for example in ultrasonic apparatuses, high pressure homogenizers, 2-, 3-, 4- or 5-roll mills, minimills, Henschel mixers, shaking mills, Ang mills, gear mills, bead mills, wet mills, sand mills, attritors, colloid mills, ultrasonic homogenizers, with Ultra Turrax stirrer and in particular by grinding, for example in 2-, 3-, 4- or 5-roll mills, minimills, shaking mills, Ang mills, gear mills, bead mills, wet mills, sand mills, colloid mills, ball mills, specifically stirred ball mills.

In a preferred embodiment ultrasonication of the Lewis Acid-donor complex, preferably the aluminum trihalide-donor complex or the alkylaluminum halide complex, or iron trihalide-donor complexes or boron halide-donor complex in an inert solvent prior to polymerization helps to improve polymerization conversion.

Furthermore, the polymerization is performed with additional use of a mono- or polyfunctional, especially mono-, di- or trifunctional, initiator which is selected from organic hydroxyl compounds, organic halogen compounds and water. It is also possible to use mixtures of the initiators mentioned, for example mixtures of two or more organic hydroxyl compounds, mixtures of two or more organic halogen compounds, mixtures of one or more organic hydroxyl compounds and one or more organic halogen compounds, mixtures of one or more organic hydroxyl compounds and water, or mixtures of one or more organic halogen compounds and water. The initiator may be mono-, di- or polyfunctional, i.e. one, two or more hydroxyl groups or halogen atoms, which start the polymerization reaction, may be present in the initiator molecule. In the case of di- or polyfunctional initiators, telechelic isobutene polymers with two or more, especially two or three, polyisobutene chain ends are typically obtained. Preferred are monofunctional initiators.

Organic hydroxyl compounds which have only one hydroxyl group in the molecule and are suitable as monofunctional initiators include especially alcohols and phenols, in particular those of the general formula R¹²—OH, in which R¹² denotes C₁- to C₂₀-alkyl radicals, especially C₁- to C₈-alkyl radicals, C₅- to C₈-cycloalkyl radicals, C₆- to C₂₀-aryl radicals, especially C₆- to C₁₂-aryl radicals, or C₇- to C₂₀-arylalkyl radicals, especially C₇- to C₁₂-arylalkyl radicals. In addition, the R¹² radicals may also comprise mixtures of the abovementioned structures and/or have other functional groups than those already mentioned, for example a keto function, a nitroxide or a carboxyl group, and/or heterocyclic structural elements.

Typical examples of such organic monohydroxyl compounds are methanol, ethanol, n-propanol, isopropanol, n-butanol, sec-butanol, isobutanol, tert-butanol, n-pentanol, n-hexanol, n-heptanol, n-octanol, 2-ethylhexanol, cyclohexanol, phenol, p-methoxyphenol, o-, m- and p-cresol, benzyl alcohol, p-methoxybenzyl alcohol, 1- and 2-phenylethanol, 1- and 2-(p-methoxyphenyl)ethanol, 1-, 2- and 3-phenyl-1-propanol, 1-, 2- and 3-(p-methoxyphenyl)-1-propanol, 1- and 2-phenyl-2-propanol, 1- and 2-(p-methoxyphenyl)-2-propanol, 1-, 2-, 3- and 4-phenyl-1-butanol, 1-, 2-, 3- and 4-(p-methoxyphenyl)-1-butanol, 1-, 2-, 3- and 4-phe-nyl-2-butanol, 1-, 2-, 3- and 4-(p-me-thoxyphenyl)-2-butanol, 9-methyl-9H-fluoren-9-ol, 1,1-diphenylethanol, 1,1-diphenyl-2-propyn-1-ol, 1,1-diphenylpropanol, 4-(1-hydroxy-1-phenyl-ethyl)benzonitrile, cyclopropyldiphenylmethanol, 1-hydroxy-1,1-diphenylpropan-2-one, benzilic acid, 9-phenyl-9-fluorenol, triphenylmethanol, diphenyl(4-pyridinyl)methanol, alpha,alpha-diphenyl-2-pyridinemethanol, 4-methoxytrityl alcohol (especially polymer-bound as a solid phase), alpha-tert-butyl-4-chloro-4′-methylbenzhydrol, cyclohexyldiphenylmethanol, alpha-(p-tolyl)-benzhydrol, 1,1,2-triphenylethanol, alpha,alpha-diphenyl-2-pyridine-ethanol, alpha,alpha-4-pyridylbenzhydrol N-oxide, 2-fluorotriphenylmethanol, triphenylpro-pargyl alcohol, 4-[(diphenyl)hydroxymethyl]benzonitrile, 1-(2,6-dimethoxy-phenyl)-2-methyl-1-phenyl-1-propanol, 1,1,2-triphenylpropan-1-ol and p-anisaldehyde carbinol.

Organic hydroxyl compounds which have two hydroxyl groups in the molecule and are suitable as bifunctional initiators are especially dihydric alcohols or diols having a total carbon number of 2 to 30, especially of 3 to 24, in particular of 4 to 20, and bisphenols having a total carbon number of 6 to 30, especially of 8 to 24, in particular of 10 to 20, for example ethylene glycol, 1,2- and 1,3-propylene glycol, 1,4-butylene glycol, 1,6-hexylene glycol, 1,2-, 1,3- or 1,4-bis(1-hydroxy-1-methylethyl)benzene (o-, m- or p-dicumyl alcohol), bisphenol A, 9,10-di-hydro-9,10-dimethyl-9,10-anthracenediol, 1,1-diphenylbutane-1,4-diol, 2-hydroxytriphenylcarbinol and 9-[2-(hydroxymethyl)-phenyl]-9-fluorenol.

Organic halogen compounds which have one halogen atom in the molecule and are suitable as monofunctional initiators are in particular compounds of the general formula R¹³—Hal in which Hal is a halogen atom selected from fluorine, iodine and especially chlorine and bromine, and R¹³ denotes C₁- to C₂₀-alkyl radicals, especially C₁- to Cs-alkyl radicals, C₅- to C₈-cycloalkyl radicals or C₇- to C₂₀-arylalkyl radicals, especially C₇- to C₁₂-arylalkyl radicals. In addition, the R¹³ radicals may also comprise mixtures of the abovementioned structures and/or have other functional groups than those already mentioned, for example a keto function, a nitroxide or a carboxyl group, and/or heterocyclic structural elements.

Typical examples of such monohalogen compounds are methyl chloride, methyl bromide, ethyl chloride, ethyl bromide, 1-chloropropane, 1-bromopropane, 2-chloropropane, 2-bromopropane, 1-chlorobutane, 1-bromobutane, sec-butyl chloride, sec-butyl bromide, isobutyl chloride, isobutyl bromide, tert-butyl chloride, tert-butyl bromide, 1-chloropentane, 1-bromopentane, 1-chloro-hexane, 1-bromohexane, 1-chloroheptane, 1-bromoheptane, 1-chlorooctane, 1-bromooctane, 1-chloro-2-ethylhexane, 1-bromo-2-ethylhexane, cyclohexyl chloride, cyclohexyl bromide, benzyl chloride, benzyl bromide, 1-phenyl-1-chloroethane, 1-phenyl-1-bromoethane, 1-phenyl-2-chloro-ethane, 1-phenyl-2-bromoethane, 1-phenyl-1-chloropropane, 1-phenyl-1-bromopropane, 1-phe-nyl-2-chloropropane, 1-phenyl-2-bromopropane, 2-phenyl-2-chloropropane, 2-phenyl-2-bromo-propane, 1-phenyl-3-chloropropane, 1-phenyl-3-bromopropane, 1-phenyl-1-chlorobutane, 1-phenyl-1-bromobutane, 1-phenyl-2-chlorobutane, 1-phenyl-2-bromobutane, 1-phenyl-3-chloro-butane, 1-phenyl-3-bromobutane, 1-phenyl-4-chlorobutane, 1-phenyl-4-bromobutane, 2-phenyl-1-chlorobutane, 2-phenyl-1-bromobutane, 2-phenyl-2-chlorobutane, 2-phenyl-2-bromobutane, 2-phenyl-3-chlorobutane, 2-phenyl-3-bromobutane, 2-phenyl-4-chlorobutane and 2-phenyl-4-bromobutane.

Organic halogen compounds which have two halogen atoms in the molecule and are suitable as difunctional initiators are, for example, 1,3-bis(1-bromo-1-methylethyl)benzene, 1,3-bis(2-chloro-2-propyl)benzene (1,3-dicumyl chloride) and 1,4-bis(2-chloro-2-propyl)benzene (1,4-dicumyl chloride).

The initiator is more preferably selected from organic hydroxyl compounds in which one or more hydroxyl groups are each bonded to an sp³-hybridized carbon atom, organic halogen compounds, in which one or more halogen atoms are each bonded to an sp³-hybridized carbon atom, and water. Among these, preference is given in particular to an initiator selected from organic hydroxyl compounds in which one or more hydroxyl groups are each bonded to an sp³-hybridized carbon atom.

In the case of the organic halogen compounds as initiators, particular preference is further given to those in which the one or more halogen atoms are each bonded to a secondary or especially to a tertiary sp³-hybridized carbon atom.

Preference is given in particular to initiators which may bear, on such an sp³-hydridized carbon atom, in addition to the hydroxyl group, the R¹², R¹³ and R¹⁴ radicals, which are each independently hydrogen, C₁- to C₂₀-alkyl, C₅-to C₈-cycloalkyl, C₆- to C₂₀-aryl, C₇- to C₂₀-alkylaryl or phenyl, where any aromatic ring may also bear one or more, preferably one or two, C₁- to C₄-alkyl, C₁- to C₄-alkoxy, C₁- to C₄-hydroxyalkyl or C₁- to C₄-haloalkyl radicals as substituents, where not more than one of the variables R¹², R¹³ and R¹⁴ is hydrogen and at least one of the variables R¹², R¹³ and R¹⁴ is phenyl which may also bear one or more, preferably one or two, C₁- to C₄-alkyl, C₁-to C₄-alkoxy, C₁- to C₄-hydroxyalkyl or C₁- to C₄-haloalkyl radicals as substituents.

For the present invention, very particular preference is given to initiators selected from water, methanol, ethanol, 1-phenylethanol, 1-(p-methoxyphenyl)ethanol, n-propanol, isopropanol, 2-phenyl-2-propanol (cumene), n-butanol, isobutanol, sec.-butanol, tert-butanol, 1-phenyl-1-chloroethane, 2-phenyl-2-chloropropane (cumyl chloride), tert-butyl chloride and 1,3- or 1,4-bis(1-hydroxy-1-methylethyl)benzene. Among these, preference is given in particular to initiators selected from water, methanol, ethanol, 1-phenylethanol, 1-(p-methoxyphenyl)ethanol, n-pro-panol, isopropanol, 2-phenyl-2-propanol (cumene), n-butanol, isobutanol, sec.-butanol, tert-butanol, 1-phenyl-1-chloroethane and 1,3- or 1,4-bis(1-hydroxy-1-methylethyl)benzene.

The molar ratio of the initiators mentioned to the isobutene monomer used in the case of homopolymerization of isobutene, or to the total amount of the polymerizable monomers used in the case of copolymerization of isobutene, based on each individual functional site of the initiator, is generally from 0.0005:1 to 0.1:1, especially 0.001:1 to 0.075:1, in particular 0.0025:1 to 0.05:1. When water is used as the sole initiator or in combination with organic hydroxyl compounds and/or organic halogen compounds as further initiators, the molar ratio of water to the isobutene monomer used in the case of homopolymerization of isobutene, or to the total amount of the polymerizable monomers used in the case of copolymerization of isobutene, is especially from 0.0001:1 to 0.1:1, in particular 0.0002:1 to 0.05:1, preferably 0.0008:1 to 0.04:1, and very preferably in particular 0.001:1 to 0.03:1.

In a preferred embodiment the amount of initiator in the monomer mixture is not more than 10 wt %, preferably not more than 7.5 wt %, more preferably not more than 5 wt %, even more preferably not more than 3 wt %, and especially not more than 2 wt %.

If water is used as the sole initiator or in combination with organic hydroxyl compounds the amount of initiator in the monomer mixture is not more than 3.2 wt %, preferably not more than 2.5 wt %, more preferably not more than 2 wt %, even more preferably not more than 1.5 wt %, and especially not more than 1 wt %.

A proportion of the initiator molecules added as organic hydroxyl or halogen compounds is incorporated into the polymer chains. The proportion (left) of polymer chains which are started by such an incorporated organic initiator molecule may be up to 100%, and is generally 5 to 90%. The remaining polymer chains arise either from water originating from traces of moisture as an initiator molecule, or from chain transfer reactions.

In a further preferred embodiment of the present invention, the polymerization is performed in the presence of 0.01 to 10 mmol, especially of 0.05 to 5.0 mmol, in particular of 0.1 to 1.0 mmol, based in each case on 1 mol of isobutene monomer used in the case of homopolymerization of isobutene, or on 1 mol of the total amount of the polymerizable monomers used in the case of copolymerization of isobutene, of a nitrogen-containing basic compound.

Such a nitrogen-containing basic compound used may be an aliphatic, cycloaliphatic or aromatic amine of the 5 general formula R¹⁴—NR¹⁵R¹⁶, or else ammonia, in which the variables R¹⁴, R¹⁵ and R¹⁶ are each independently hydrogen, C₁- to C₂₀-alkyl radicals, especially C₁- to C₈-alkyl radicals, C₅- to C₈-cycloalkyl radicals, C₆- to C₂₀-aryl radicals, especially C₆- to C₁₂-aryl radicals, or C₇- to C₂₀-arylalkyl radicals, especially C₇- to C₁₂-arylalkyl radicals.

When none of these variables is hydrogen, the amine is a tertiary amine. When one of these variables is hydrogen, the amine is a secondary amine. When two of these variables is hydrogen, the amine is a primary amine. When all these variables are hydrogen, the amine is ammonia.

Typical examples of such amines of the general formula R¹⁴—NR¹⁵R¹⁶ are methylamine, ethylamine, n-propylamine, isopropylamine, n-butylamine, tert-butylamine, sec-butylamine, isobutyl-amine, tert-amylamine, n-hexylamine, n-heptylamine, n-octylamine, 2-ethylhexylamine, cyclopentylamine, cyclohexylamine, aniline, dimethylamine, diethylamine, di-n-propylamine, diisopropylamine, di-n-butylamine, di-tert-butylamine, di-sec-butylamine, diisobutylamine, di-tert-amylamine, di-n-hexylamine, di-n-heptylamine, di-n-octylamine, di-(2-ethylhexyl)amine, dicyclopentylamine, dicyclohexylamine, diphenylamine, trimethylamine, triethylamine, tri-n-propyl-amine, tri-isopropylamine, tri-n-butylamine, tri-tert-butylamine, tri-sec-butylamine, tri-isobutyl-amine, tri-tert-amylamine, tri-n-hexylamine, tri-n-heptylamine, tri-n-octylamine, tri-(2-ethylhexyl)-amine, tricyclopentylamine, tricyclohexylamine, triphenylamine, dimethylethylamine, methyl-n-butylamine, N-methyl-N-phenylamine, N,N-dimethyl-N-phenylamine, N-methyl-N,N-diphenylamine or N-methyl-N-ethyl-N-n-butylamine.

In addition, such a nitrogen-containing basic compound used may also be a compound having a plurality of, especially having two or three, nitrogen atoms and having 2 to 20 carbon atoms, where these nitrogens may each independently bear hydrogen atoms or aliphatic, cycloaliphatic or aromatic substituents. Examples of such polyamines are 1,2-ethylenediamine, 1,3-propylene-diamine, 1,4-butylenediamine, diethylenetriamine, N-methyl-1,2-ethylenediamine, N,N-dimethyl-1,2-ethylenediamine, N,N′-dimethyl-1,2-ethylenediamine or N,N-dimethyl-1,3-propylenediamine.

However, a suitable nitrogen-containing basic compound of this kind is especially a saturated, partly unsaturated or unsaturated nitrogen-containing five-membered or six-membered heterocyclic ring which comprises one, two or three ring nitrogen atoms and may have one or two further ring heteroatoms from the group of oxygen and sulphur and/or hydrocarbyl radicals, especially C₁- to C₄-alkyl radicals and/or phenyl, and/or functional groups or heteroatoms as substituents, especially fluorine, chlorine, bromine, nitro and/or cyano, for example pyrrolidine, pyrrole, imidazole, 1,2,3- or 1,2,4-triazole, oxazole, thiazole, piperidine, pyrazane, pyrazole, pyridazine, pyrimidine, pyrazine, 1,2,3-, 1,2,4- or 1,2,5-triazine, 1,2,5-oxathiazine, 2H-1,3,5-thiadiazine or morpholine.

However, a very particularly suitable nitrogen-containing basic compound of this kind is pyridine or a derivative of pyridine (especially a mono-, di- or tri-C₁- to C₄-alkyl-substituted pyridine) such as 2-, 3-, or 4-methylpyridine (picolines), 2,3-, 2,4-, 2,5-, 2,6-, 3,4-, 3,5- or 3,6-dimethylpyridine (lutidines), 2,4,6-trimethylpyridine (collidine), 2-, 3, - or 4-tert-butylpyridine, 2-tert-butyl-6-methyl-pyridine, 2,4-, 2,5-, 2,6- or 3,5-di-tert-butylpyridine or else 2-, 3, - or 4-phenylpyridine.

It is possible to use a single nitrogen-containing basic compound or mixtures of such nitrogen-containing basic compounds.

The polymerization of the isobutene or of the isobutenic starting material generally proceeds spontaneously when the Lewis Acid, preferably the Lewis Acid-donor complex, more preferably the boron trihalide-donor complex, aluminum trihalide-donor complex or the alkylaluminum halide complex, especially the alkyl aluminum dichloride-donor complex or dialkyl aluminum chloride-donor complex, is contacted with the isobutene or the isobutenic monomer mixture at the desired reaction temperature. The procedure here may be to initially charge the monomers, optionally in the diluent, to bring it to reaction temperature and then to add the Lewis Acid-donor complex, preferably the boron trihalide-donor complex, aluminum trihalide-donor complex or the alkylaluminum halide complex, especially the alkyl aluminum dichloride-donor complex or dialkyl aluminum chloride-donor complex. The procedure may also be to initially charge the Lewis Acid-donor complex, preferably the boron trihalide-donor complex, aluminum trihalide-donor complex or the alkylaluminum halide complex, especially the alkyl aluminum dichloride-donor complex or dialkyl aluminum chloride-donor complex, optionally in the diluent, and then to add the monomers. In that case, the start of polymerization is considered to be that time at which all reactants are present in the reaction vessel.

To prepare isobutene copolymers, the procedure may be to initially charge the monomers, optionally in the diluent, and then to add the Lewis Acid-donor complex, preferably the boron trihalide-donor complex, aluminum trihalide-donor complex or the alkylaluminum halide complex, especially the alkyl aluminum dichloride-donor complex or dialkyl aluminum chloride-donor complex. The reaction temperature can be established before or after the addition of the Lewis Acid-donor complex, preferably boron trihalide-donor complex, the aluminum trihalide-donor complex or the alkylaluminum halide complex, especially of the alkyl aluminum dichloride-donor complex or dialkyl aluminum chloride-donor complex. The procedure may also be first to initially charge only one of the monomers, optionally in the diluent, then to add the Lewis Acid-donor complex, preferably the boron trihalide-donor complex, aluminum trihalide-donor complex or the alkylaluminum halide complex, especially the alkyl aluminum dichloride-donor complex or dialkyl aluminum chloride-donor complex, and to add the further monomer(s) only after a certain time, for example when at least 60%, at least 80% or at least 90% of the monomer has been converted. Alternatively, the Lewis Acid-donor complex, preferably the boron trihalide-donor complex, aluminum trihalide-donor complex or the alkylaluminum halide complex, especially the alkyl aluminum dichloride-donor complex or dialkyl aluminum chloride-donor complex, can be initially charged, optionally in the diluent, then the monomers can be added simultaneously or successively, and then the desired reaction temperature can be established. In that case, the start of polymerization is considered to be that time at which the Lewis Acid-donor complex, preferably the boron trihalide-donor complex, aluminum trihalide-donor complex or the alkylaluminum halide complex, especially the alkyl aluminum dichloride-donor complex or dialkyl aluminum chloride-donor complex, and at least one of the monomers are present in the reaction vessel.

In addition to the batchwise procedure described here, the polymerization in the process according to the invention can also be configured as a continuous process. In this case, the feedstocks, i.e. the monomer(s) to be polymerized, optionally the diluent and optionally the Lewis Acid-donor complex, preferably the boron trihalide-donor complex, aluminum trihalide-donor complex or the alkylaluminum halide complex, especially the alkyl aluminum dichloride-donor complex or dialkyl aluminum chloride-donor complex, are supplied continuously to the polymerization reaction, and reaction product is withdrawn continuously, such that more or less steady-state polymerization conditions are established in the reactor. The monomer(s) to be polymerized can be supplied as such, diluted with a diluent or solvent, or as a monomer-containing hydrocarbon stream.

The Lewis Acid-donor complex, preferably the boron trihalide-donor complex, aluminum trihalide-donor complex effective as a polymerization catalyst or the alkylaluminum halide complex, especially alkyl aluminum dichloride-donor complex or dialkyl aluminum chloride-donor complex, is generally present in dissolved, dispersed or suspended form in the polymerization medium. Supporting of the Lewis Acid-donor complex, preferably of the boron trihalide-donor complex, aluminum trihalide-donor complex or of the alkylaluminum halide complex, especially of alkyl aluminum dichloride-donor complex or dialkyl aluminum chloride-donor complex, on customary support materials is also possible. Suitable reactor types for the polymerization process of the present invention are typically stirred tank reactors, loop reactors and tubular reactors, but also fluidized bed reactors, stirred tank reactors with or without solvent, fluid bed reactors, continuous fixed bed reactors and batchwise fixed bed reactors (batchwise mode).

In principle, all types of discontinuous or continuously operated reactors suitable for such liquid-phase polymerizations can be used as polymerization reactors for the process according to the invention, such as stirred tanks, stirred tank cascades, tube reactors or loop reactors. If polymerization is carried out according to the method according to the invention at or above the boiling temperature of any inert diluent or monomer to be polymerized, it is preferably carried out in pressure vessels, for example in autoclaves or in pressure reactors.

Furthermore, it is important that polymerizable compounds are moved in containers, for example by stirring, natural circulation or pumping around (forced circulation).

In the process according to the invention, the boron trihalide-donor complex or aluminum trihalide-donor complex effective as a polymerization catalyst or the alkylaluminum halide complex, or the iron trihalide-donor complex, or the gallium trihalide-donor complex, or the titanium tetrahalide-donor complex, or the zinc dihalide-donor complex, or the tin dihalide-donor complex, or the tin tetrahalide-donor complex or the boron trihalide-donor complex, especially the alkyl aluminum dichloride-donor complex or dialkyl aluminum chloride-donor complex, is generally used in such an amount that the molar ratio of the metal in the Lewis Acid donor complex, preferably the aluminum in the aluminum trihalide-donor complex or alkylaluminum halide complex, especially in the alkyl aluminum dichloride-donor complex or dialkyl aluminum chloride-donor complex, to isobutene in the case of homopolymerization of isobutene, or to the total amount of the polymerizable monomers used in the case of copolymerization of isobutene, is in the range from 1:5 to 1:5000, preferably from 1:10 to 1:5000, especially 1:15 to 1:1000, in particular 1:20 to 1:250.

In a preferred embodiment of the present invention the reaction conditions are chosen that the conversion of the monomers is at least 80%, preferably at least 85%, and more preferably at least 90%.

According to the invention the polymerization is carried out as a polymerization in bulk or in solution.

The polymerization in the process according to the invention is preferably performed in the presence of an inert diluent. The inert diluent used should be suitable for reducing the increase in the viscosity of the reaction solution which generally occurs during the polymerization reaction to such an extent that the removal of the heat of reaction which evolves can be ensured. Suitable diluents are those solvents or solvent mixtures which are inert toward the reagents used. Suitable diluents are, for example, aliphatic hydrocarbons such as n-butane, n-pentane, n-hexane, n-heptane, n-octane and isooctane, cycloaliphatic hydrocarbons such as cyclopentane and cyclohexane, aromatic hydrocarbons such as benzene, toluene and the xylenes, and halogenated hydrocarbons, especially halogenated aliphatic hydrocarbons, such as methyl chloride, dichloromethane and trichloromethane (chloroform), 1,1-dichloroethane, 1,2-dichloroethane, trichloroethane and 1-chlorobutane, and also halogenated aromatic hydrocarbons and alkylaromatics halogenated in the alkyl side chains, such as chlorobenzene, monofluoromethylbenzene, difluoromethylbenzene and trifluoromethylbenzene, and mixtures of the aforementioned diluents. The diluents used, or the constituents used in the solvent mixtures mentioned, are also the inert components of isobutenic C4 hydrocarbon streams. A non-halogenated solvent is preferred over the list of halogenated solvents.

The inventive polymerization may be performed in a halogenated hydrocarbon, especially in a halogenated aliphatic hydrocarbon, or in a mixture of halogenated hydrocarbons, especially of halogenated aliphatic hydrocarbons, or in a mixture of at least one halogenated hydrocarbon, especially a halogenated aliphatic hydrocarbon, and at least one aliphatic, cycloaliphatic or aromatic hydrocarbon as an inert diluent, for example a mixture of dichloromethane and n-hexane, typically in a volume ratio of 10:90 to 90:10, especially of 50:50 to 85:15. Prior to use, the diluents are preferably freed of impurities such as water, carboxylic acids or mineral acids, for example by adsorption on solid adsorbents such as activated carbon, molecular sieves or ion exchangers.

In a preferred embodiment, the inventive polymerization is performed in halogen-free aliphatic or especially halogen-free aromatic hydrocarbons, especially toluene. For this embodiment, water in combination with the organic hydroxyl compounds mentioned and/or the organic halogen compounds mentioned, or especially as the sole initiator, have been found to be particularly advantageous.

In another preferred embodiment, the inventive polymerization is performed in halogen-free aliphatic or cycloaliphatic, preferably aliphatic hydrocarbons, especially hexane, pentane, heptane, cyclohexane, cyclopentane, and mixtures comprising them.

The polymerization in the process according to the invention is preferably performed under substantially aprotic and especially under substantially anhydrous reaction conditions. Substantially aprotic and substantially anhydrous reaction conditions are understood to mean that, respectively, the content of protic impurities and the water content in the reaction mixture are less than 50 ppm and especially less than 5 ppm. In general, the feedstocks will therefore be dried before use by physical and/or chemical measures. More particularly, it has been found to be useful to admix the aliphatic or cycloaliphatic hydrocarbons used as solvents, after customary prepurification and predrying with an organometallic compound, for example an organolithium, organomagnesium or organoaluminum compound, in an amount which is sufficient to substantially remove the water traces from the solvent. The solvent thus treated is then preferably condensed directly into the reaction vessel. It is also possible to proceed in a similar manner with the monomers to be polymerized, especially with isobutene or with the isobutenic mixtures. Drying with other customary desiccants such as molecular sieves or predried oxides such as aluminum oxide, silicon dioxide, calcium oxide or barium oxide is also suitable. The halogenated solvents for which drying with metals such as sodium or potassium or with metal alkyls is not an option are freed of water or water traces with desiccants suitable for that purpose, for example with calcium chloride, phosphorus pentoxide or molecular sieves. It is also possible in an analogous manner to dry those feedstocks for which treatment with metal alkyls is likewise not an option, for example vinylaromatic compounds. Even if some or all of the initiator used is water, residual moisture should preferably be very substantially or completely removed from solvents and monomers by drying prior to reaction, in order to be able to use the water initiator in a controlled, specified amount, as a result of which greater process control and reproducibility of the results are obtained.

To stop the reaction, the reaction mixture is preferably deactivated, for example by adding a protic compound, especially by adding water, alcohols such as methanol, ethanol, n-propanol and isopropanol or mixtures thereof with water, or by adding an aqueous base, for example an aqueous solution of an alkali metal or alkaline earth metal hydroxide such as sodium hydroxide, potassium hydroxide, magnesium hydroxide or calcium hydroxide, an alkali metal or alkaline earth metal carbonate such as sodium, potassium, magnesium or calcium carbonate, or an alkali metal or alkaline earth metal hydrogencarbonate such as sodium, potassium, magnesium or calcium hydrogencarbonate.

Depending on the reaction conditions, such as reaction temperature, amount of Lewis Acid, ratio of Lewis Acid and donor compound, residence time, mixing conditions etc., the process according to the invention serves to prepare polyisobutene with different molecular weight and different reactivity:

Depending on the molecular weight and on the content of terminal double bond the polyisobutene is referred to as
High molecular polyisobutene with a number-average molecular weight Mₙ of more than 100000 g/mol
Medium molecular polyisobutene with a number-average molecular weight Mₙ of more than 10000 and up to 100000 g/mol
Low molecular polyisobutene with a number-average molecular weight Mₙ of more than 350 and up to 10000 g/mol
Low molecular polyisobutene is usually referred to as highly reactive with a content of terminal (alpha-) double bonds of at least 50 mol %, preferably at least 60, more preferably at least 70, even more preferably at least 80, and especially at least 85 mol %. The content of such alpha-double bonds may be up to 100, up to 98, up to 97 or up to 95 mol %
Medium molecular polyisobutene is usually referred to as highly reactive with a content of terminal (alpha-) double bonds of 10 to 60 mol %, preferably 15 to 55 mol %, more preferably 20 to 50 mol %, even more preferably 25 to 50 mol %, and especially at least 25 to 45 mol %.

The process according to the invention also serves to prepare high-reactivity isobutene copolymers which are formed from isobutene and at least one vinylaromatic monomer, especially styrene, and have a content of terminal vinylidene double bonds (a-double bonds) and other reactive double bonds, such as p-double bonds per polyisobutene chain end of at least 60, preferably at least 70 mol %, preferably of at least 75 mol %. To prepare such copolymers of isobutene and at least one vinylaromatic monomer, especially styrene, isobutene or an isobutenic hydrocarbon cut is copolymerized with the at least one vinylaromatic monomer in a weight ratio of isobutene to vinylaromatic of 5:95 to 95:5, especially of 30:70 to 70:30.

The high-reactivity isobutene homo- or copolymers prepared by the process according to the invention and specifically the isobutene homopolymers preferably have a polydispersity (PDI=M_{w}/Mₙ) of 1.05 to less than 3.5, preferably of 1.05 to less than 3.0, preferably of 1.05 to less than 2.5, preferably of 1.05 to 2.3, more preferably of 1.05 to 2.0 and especially of 1.1 to 1.85. Typical PDI values in the case of an optimal process regime are 1.2 to 1.7.

The method according to the invention is operated at different reaction temperatures depending on the desired molar weight of the polyisobutene:

In a preferred embodiment of the present invention, when a polyisobutene with a number average molecular weight Mn of 350 to 10000, preferably 500 to 5000 is targeted, the reaction temperature in the reaction mixture is preferably from −5 to +25° C.

In particular, this homo- or copolymer is a highly reactive polyisobutene with a content of terminal vinylidene groups of at least 70 mol %, preferably at least 80 mol %, preferably at least 90 mol %.

In an alternative preferred embodiment of the present invention, when a number average molecular weight Mn of 10000 to 100000 is targeted, the reaction temperature in the reaction mixture is preferably from −30 to −10° C.

In an alternative preferred embodiment of the present invention, when a number average molecular weight Mn of at least 100000 to 1000000 is sought, the reaction temperature in the reaction mixture is preferably from −90 to −50° C.

As a rule, the average residence time in the reaction system without mixing, e.g. by circulation, stirring or backmixing, should be less than 2 hours, preferably less than 90 minutes and preferably less than 60 minutes.

Polymerization is usually carried out at a pressure of 700 mbar to 20 bar, especially at a pressure of 1 bar to 10 bar, especially at a pressure of 1.2 bar to 7 bar. Overpressure is usually advantageous with the C₄ hydrocarbon mixture used and with some inert diluents that may be used.

Another object of the present invention is the polyisobutene obtainable preferably obtained by the process according to the present invention.

Polyisobutene, especially highly reactive polyisobutene of low or medium molecular weight serves as a valuable starting material for further derivatisation, such as Friedel-Crafts alkylation of aromatic compounds, ene reaction with maleic anhydride, epoxidation or hydroformylation which in turn serve as starting material for further reaction, e.g. reaction of polyisobutenyl succinic anhydride with amines, or in the case of hydroformylated polyisobutene subsequent hydrogenation or amination to polyisobutene amine. These products find use e.g. as fuel additives.

Medium and high molecular weight polyisobutene not exhibiting a high reactivity is less susceptible to weathering and more stable against oxidation or thermal degradation. Therefore, compositions comprising such polyisobutenes are especially useful in sealants, adhesives, coatings or roofings.

### EXAMPLES

To illustrate the invention, various catalyst samples were synthesized and tested in a laboratory catalysis apparatus under the process conditions of a Guerbet reaction, with the condensation of methanol/ethanol in the gas phase to form i-butanol being examined. Precipitation processes and impregnation processes were used to synthesize the catalyst. A summary of the individual illustrative samples and their chemical composition is given in Table 1.

### Production of the Illustrative Samples

### Process by Means of Compacting and Impregnation

A commercially available finely divided hydrotalcite powder was firstly processed in a granulator (in the present case a roller compactor) to give a coarse-grained granular material. The coarse-grained granular material was rubbed through a sieve having a mesh opening of 1 mm and finely divided material was sieved out (500 μm mesh opening), giving a compacted hydrotalcite powder having a particle size in the range 0.5-1 mm. Pural® MG 70 (from Sasol) was used as commercially available hydrotalcite. Pural® MG 70 is aluminum magnesium hydroxy carbonate. It has a 70:30 MgO:Al₂O₃ ratio. A roller compactor from Powtec (model RC 100×30) was used as granulator. In the compaction operation, 250 g of finely divided powder were treated in a plurality of passes using corrugated rollers and a pressing pressure of 250 bar in each case. The rollers had a rotational speed of 5 rpm. The granulator was equipped with a sieve insert having a mesh opening of 1.6 mm.

The compacted hydrotalcite powder had a water absorption capacity (or liquid absorption capacity) of 0.46 ml/g and the loss on ignition of the compacted hydrotalcite powder, which was determined at 600° C., was 44.7% by weight. From one batch to the next, the water absorption capacity and the loss on ignition of the sample produced in each case could be subject to small deviations, which was then taken into account in the appropriate way for the addition of impregnation solution.

The application of the promoter elements to the compacted hydrotalcite powder was effected by means of impregnation. Firstly, 12 g each of the compacted hydrotalcite powder were placed in individual porcelain dishes having a diameter of 8 cm. The metal salt solutions were then added in the previously determined concentrations and amounts of liquid to the respective porcelain dishes in order to apply the desired target amount of metal species and not to exceed the liquid absorption capacity of the powder. This impregnation process on the dry powder is an incipient wetness process with complete filling of the pores. The porcelain dishes filled with powder samples were kept in motion or rotated during and after addition of the impregnation solution by means of a laboratory shaking machine, namely at a speed of 1000 rpm.

### Illustrative Sample B1

In the production of illustrative sample B1, a solution comprising Cu(NO₃)₂2.5H₂O was used for application of the promoter. To produce the impregnation solution, 0.03 ml of Cu(NO₃)₂2.5H₂O solution (admixed with 5.387 g of water were mixed and added to the initially charged 8 g hydrotalcite sample. To effect mixing or aging of the impregnated hydrotalcite sample, the porcelain dish filled with hydrotalcite sample was kept in motion for another 30 minutes at room temperature. The illustrative samples B2 to B7 shown in Table 1 were produced by the same method, with the promoter component(s) being varied in terms of composition.

To dry the sample, the porcelain dish charged with the sample was firstly stored at 80° C. in a convection drying oven for 16 hours. The sample was subsequently calcined in a calcination furnace (model LH120/12 from Nabertherm). For the calcination, the sample in the furnace was firstly heated to 250° C. and maintained at 250° C. for 4 hours. A stream of air of 6 I/min was introduced into the furnace during the entire calcination phase and cooling phase. In the heating phases, a heating rate of 5 K/min was used.

The calcined sample material was subjected to sieving in order to obtain the sample having a particle size of 0.5-1 mm. For this purpose, sieves having a mesh opening of 1 mm and 0.5 mm were used and the powder samples were firstly distributed over the area of the coarse sieve using the edge of a spatula and the fines were subsequently removed through the fine sieve.

TABLE 1
Summary overview of the illustrative samples which were used for
the catalytic tests, and also their chemical composition in respective
of the promoter elements and the starting materials used for the
synthesis and the basis of the support oxide. The abbreviation
PMG 70 means that Pural ® MG70 from Sasol was used for
synthesizing the catalyst. The samples were all calcined at 250° C.

| | Catalyst | Cu [% | Ru [% | Ir [% | Basis of the |
|---|---|---|---|---|---|
| | No. | by weight] | by weight] | by weight] | support oxide |
| 1 | B1 | 0.1 | 0 | 0 | PMG 70 |
| 2 | B2 | 1.0 | 0 | 0 | PMG 70 |
| 3 | B3* | 10.0 | 0 | 0 | PMG 70 |
| 4 | B4 | 5 | 0.1 | 0 | PMG 70 |
| 5 | B5 | 5 | 0 | 0.1 | PMG 70 |
| 6 | B6* | 0 | 0.1 | 0 | PMG 70 |
| 7 | B7 | 0 | 0 | 0.1 | PMG 70 |

*Comparative catalyst.

### Catalytic Studies

### Condensation of Different Alcohol Components (Methanollethanol)

The results of the catalytic tests for the condensation of different alcohol components preferentially condensation of ethanol and methanol are shown in tables 2 to 5. The catalytic tests were, with only a few exceptions, in each case carried out on 0.5 and 1 ml of the pulverulent illustrative samples, using a crushed material fraction having a particle size in the range from 0.5 to 1 mm for this purpose. To prepare for the studies, the samples were positioned on a catalyst support grid or on a bed of inert particles in tube reactors, the loaded reactors were installed in 16-fold high throughput catalysis testing apparatus and the samples comprised therein were subject to the test procedures. The test procedures generally provided for the samples to be subjected to activation before the catalysis experiments. Activation of catalysts was performed at 400° C. under 20 vol % H₂ in inert gas for 10 h.

In addition, all catalysts were also subjected to a conditioning treatment at 250° C. under the flow of 1 vol % ethanol for 24 h. All catalysis experiments were carried out at temperatures within the range from 250 to 325° C. A gas chromatograph coupled with a mass spectrometer (a GC-MS from Agilent) and equipped with FID and TCD was used for analyzing the product gas stream.

Another important finding was that the catalytic properties of the catalysts in respect of the process of the invention for condensing alcohols are significantly improved when the process is preceded by an activation and/or conditioning process.

An overview of a first series of experiments and the results achieved therein is shown in table 2. In the experiments shown there, the feed gas flow was selected so that the GHSV was 1000 h⁻¹.

An inert gas stream composed of nitrogen and argon loaded with ethanol, and methanol vapor was used as feed gas stream. The proportion of argon was kept constant in all experiments and was 5% by volume. The ethanol content was in each case set in the various experiments and was varied from 1 vol % to 20 vol % whereas methanol content was kept constant at 20 vol %.

Table 2 shows the results of the catalytic tests on the reaction of EtOH and MeOH at a reaction temperature of 325° C. using a feed comprising the EtOH and MeOH in a molar ratio of 1:20 in the presence of 20% by volume of H₂ at GHSV 1000 h⁻¹ at 7 bar reaction pressure.

| | | EtOH | MeOH | IsoBuOH | PrOH | Isobutanal | Propanal |
|---|---|---|---|---|---|---|---|
| | Catalyst | Conv (%) | Conv (%) | Sel (%) | Sel (%) | Sel(%) | Sel (%) |
| Example No. | | | | | | | |
| 1 | B1 | 90 | 25 | 41 | 3 | 2 | 0 |
| 2 | B2 | 100 | 57 | 24 | 0 | 10 | 0 |
| 3 | B4 | 100 | 73 | 13 | 1 | 5 | 0 |
| 4 | B5 | 100 | 63 | 20 | 1 | 8 | 0 |
| Comp. Ex. | | | | | | | |
| 1 | B6 | 25 | 46 | 5 | 5 | 0 | 0 |
| 2 | B7 | 53 | 28 | 20 | 9 | 16 | 0 |
| 3 | B3 | 95 | 86 | 14 | 2 | 5 | 0 |

Table 3 shows the results of the catalytic tests on the reaction of EtOH and MeOH at a reaction temperature of 325° C. using a feed comprising the EtOH and MeOH in a molar ratio of 1:20 in the presence of 20% by volume of H₂ at GHSV 2000 h⁻¹ at 7 bar reaction pressure.

| | | EtOH | MeOH | IsoBuOH | PrOH | Isobutanal | Propanal |
|---|---|---|---|---|---|---|---|
| | Catalyst | Conv (%) | Conv (%) | Sel (%) | Sel (%) | Sel(%) | Sel (%) |
| Example No. | | | | | | | |
| 5 | B1 | 60 | 11 | 28 | 15 | 0 | 4 |
| 6 | B2 | 98 | 37 | 31 | 0 | 0 | 0 |
| 7 | B4 | 100 | 58 | 18 | 0 | 0 | 0 |
| 8 | B5 | 100 | 49 | 24 | 0 | 0 | 0 |
| Comp. Ex. | | | | | | | |
| 4 | B6 | 28 | 21 | 9 | 9 | 0 | 0 |
| 5 | B7 | 42 | 13 | 20 | 15 | 0 | 0 |
| 6 | B3 | 95 | 70 | 16 | 2 | 0 | 1 |

Table 4 shows the results of the catalytic tests on the reaction of EtOH and MeOH at a reaction temperature of 325° C. using a feed comprising the EtOH and MeOH in a molar ratio of 1:20 at GHSV 1000 h⁻¹ at 7 bar reaction pressure.

| | | EtOH | MeOH | IsoBuOH | PrOH | Isobutanal | Propanal |
|---|---|---|---|---|---|---|---|
| | Catalyst | Conv (%) | Conv (%) | Sel (%) | Sel (%) | Sel(%) | Sel (%) |
| Example No. | | | | | | | |
| 9 | B1 | 34 | 10 | 16 | 15 | 11 | 4 |
| 10 | B2 | 96 | 26 | 22 | 2 | 41 | 1 |
| 11 | B4 | 100 | 55 | 19 | 0 | 15 | 0 |
| 12 | B5 | 100 | 45 | 22 | 0 | 22 | 0 |
| Comp. Ex. | | | | | | | |
| 7 | B6 | 1 | 31 | 6 | 7 | 0 | 0 |
| 8 | B7 | 34 | 18 | 19 | 15 | 3 | 0 |
| 9 | B3 | 100 | 39 | 25 | 0 | 27 | 0 |

Table 5 shows the results of the catalytic tests on the reaction of EtOH and MeOH at a reaction temperature of 325° C. using a feed comprising the EtOH and MeOH in a molar ratio of 1:20 at GHSV 2000 h⁻ at 7 bar reaction pressure.

| | | EtOH | MeOH | IsoBuOH | PrOH | Isobutanal | Propanal |
|---|---|---|---|---|---|---|---|
| | Catalyst | Conv (%) | Conv (%) | Sel (%) | Sel (%) | Sel(%) | Sel (%) |
| Example No. | | | | | | | |
| 13 | B1 | 16 | 5 | 12 | 15 | 8 | 3 |
| 14 | B2 | 67 | 12 | 9 | 10 | 43 | 10 |
| 15 | B4 | 90 | 30 | 19 | 3 | 28 | 2 |
| 16 | B5 | 91 | 21 | 17 | 3 | 43 | 3 |
| Comp. Ex. | | | | | | | |
| 10 | B6 | 15 | 11 | 15 | 18 | 2 | 0 |
| 11 | B7 | 26 | 7 | 15 | 16 | 6 | 2 |
| 12 | B5 | 91 | 21 | 17 | 3 | 43 | 3 |

### Equilibration

The Cu catalysts with high Cu content (1.0 and 10% by weight) were equilibrated under mixed Guerbet conditions (temperature of 325° C. using a feed comprising the EtOH and MeOH in a molar ratio of 1:20 in the presence of 20% by volume of H₂ at 7 bar reaction pressure) over 100 h time on stream. The selectivity of Cu catalysts changed from CO/CO₂ to Guerbet intermediates such as C₃/C₄ aldehydes and C₃/C₄ alcohols. This equilibration phase also led to the complete disappearance of CO₂ from the product spectrum over time on stream.

For example, a catalyst containing 0.1% by weight copper was more selective to C₄/C₃ products at the beginning and a catalyst containing 1.0% by weight copper produced more CO/CO₂, as with time on stream, the catalyst containing 1.0% by weight copper showed improved selectivity almost the same as the catalyst containing 0.1% by weight copper and also good conversion of EtOH and MeOH.

In summary, the copper comprising catalysts of the present invention were identified to successfully catalyze mixed Guerbet condensation of ethanol and methanol to isobutanol. The catalysts exhibited approximately 75% selectivity (carbon based) to C—C coupling products of mixed Guerbet reaction producing mainly isobutanol, isobutyraldehyde, propanol and propionaldehyde. A 20:1 (MeOH:EtOH) ratio was suitable for highest isobutanol selectivity. With changing MeOH:EtOH ratio, the nature of product distribution could be changed to produce mixture product stream of C₃-C₄ alcohols and aldehydes. Another standout factor could be attributed to the positive effect of high reaction pressure (7 bar) compared to that of literature. The process of the present invention represents a sustainable method to produce renewable isobutanol.

1. A process for manufacturing of polyisobutene, comprising:
condensation of methanol and a second alcohol in the gas phase yielding an isobutanol-containing reaction mixture,
dehydrating isobutanol to isobutene,
followed by polymerization of the thus obtained isobutene to polyisobutene.

2. The process according to claim 1, wherein the second alcohol is ethanol.

3. The process according to claim 2, wherein a proportion of methanol in a feed gas stream is from five to twenty times higher than a proportion of ethanol.

4. The process according to claim 1, wherein the second alcohol is n-propanol.

5. The process according to claim 4, wherein the proportion of methanol in the feed gas stream is from five to ten times higher than a proportion of n-propanol.

6. The process according to claim 1, wherein the isobutanol-containing reaction mixture is purified by distillation to yield an isobutanol-enriched fraction which is used in the subsequent dehydration.

7. The process according to claim 1, wherein dehydration of isobutanol to isobutene takes place in the gas phase at a temperature of from 108 to 600° C. and a pressure of from 0.05 to 1 MPa in the presence of a catalyst.

8. The process according to claim 1, wherein the isobutene-containing reaction mixture is purified by distillation to yield an isobutene-enriched fraction which is used in the subsequent polymerization.

9. The process according to claim 1, wherein the polymerization of isobutene to polyisobutene takes place in the presence of at least one Lewis Acid and in the presence of at least one initiator.

10. The process according to claim 9, wherein the at least one Lewis Acid is selected from the group consisting of aluminum trihalide, alkylaluminum halide, iron trihalide, a gallium trihalide, a titanium tetrahalide, a zinc dihalide, a tin dihalide, a tin tetrahalide, and a boron trihalide.

11. The process according to claim 9, wherein the at least one Lewis Acid is deployed together with a donor forming a Lewis-Acid-donor complex, wherein the donor is at least one organic compound comprising at least one oxygen or nitrogen atom with at least one lone electron pair.

12. The process according to claim 1, wherein the condensation of methanol and a second alcohol in the gas phase is conducted in the presence of a catalyst for condensing alcohols, which comprises support material in contact with copper as promoter, wherein
a) the support material comprises hydrotalcite-like compounds,
b) the proportion of copper promoter is in a range of from 0.05 to 5.0% by weight,
c) the support material has an Mg/Al ratio in a range of from 90/10 to 70/30, where the Mg/Al ratio is based on the weight of the respective oxides, wherein the catalyst may optionally comprise at least one further promoter element selected from the group consisting of Pt, Rh, Ru, Pd, Co, Ni, Pd, Cu, Ag and Au and a content of the at least one further promoter element is in a range of 0.01 to 2% by weight, with the proviso that an amount of copper promoter is greater than an amount of the at least one further promoter element.

13. The process according to claim 12, wherein the catalyst comprises at least one further promoter element from the group consisting of Ru and Ir and the content of the at least one further promoter element is in the range of from 0.01 to 2.0% by weight.

14. The process according to claim 12, wherein the catalyst is obtainable by a process comprising:
d) bringing into contact a support material comprising a hydrotalcite-like compound with a promoter source,
e) producing an intimate mixture of support material comprising a hydrotalcite-like compound and the promoter source,
f) thermally treating the intimate mixture of support material comprising a hydrotalcite-like compound and the promoter source, with the thermal treatment comprising a calcination process at a temperature in a range 200-1000° C.

15. The process according to claim 1, comprising:
bringing an alcohol-comprising feed gas stream comprising methanol and a second alcohol into contact with a catalyst for condensing alcohols, which comprises support material in contact with copper as promoter, wherein
a) the support material comprises hydrotalcite-like compounds,
b) the proportion of copper promoter is in a range of from 0.05 to 5.0% by weight,
c) the support material has an Mg/Al ratio in a range of from 90/10 to 70/30, where the Mg/Al ratio is based on the weight of the respective oxides, wherein the catalyst may optionally comprise at least one further promoter element selected from the group consisting of Pt, Rh, Ru, Pd, Co, Ni, Pd, Cu, AR and Au and a content of the at least one further promoter element is in a range of 0.01 to 2% by weight, with the proviso that an amount of copper promoter is greater than an amount of the at least one further promoter element.

16. The process according to claim 9, wherein the at least one Lewis Acid is deployed together with a donor forming a Lewis-Acid-donor complex, wherein the donor is selected from the group consisting of organic compounds with at least one ether function, organic compounds with at least one carboxylic ester function, organic compounds with at least one aldehyde function, organic compounds with at least one keto function, and organic compounds with at least one nitrogen containing heterocyclic ring.

17. The process according to claim 12, wherein the catalyst is obtainable by a process comprising:
d) bringing into contact a support material comprising a hydrotalcite precursor material with a promoter source,
e) producing an intimate mixture of the support material comprising the hydrotalcite precursor material and the promoter source,
f) thermally treating the intimate mixture of the support material comprising the hydrotalcite precursor material and the promoter source, with the thermal treatment comprising a calcination process at a temperature in a range of 200-1000° C.
